(19) United States
(12) Patent Application Publication
(10) Pub. No.: US 2025/0230437 A1
(43) Pub. Date: Jul. 17, 2025

(54) Novel systems for nucleic acid regulation

(71) Applicant: uniQure biopharma B.V., Amsterdam (NL)

(72) Inventors: Irena Bockaj, Amsterdam (NL); Morgane Sarah Wartel, Amsterdam (NL); Sebastian Niklas Kieper, Amsterdam (NL); Astrid Valles Sánchez, Amsterdam (NL); Seyda Acar Broekmans, Amsterdam (NL); Melvin Maurice Evers, Amsterdam (NL)

(73) Assignee: uniQure biopharma B.V., Amsterdam (NL)

(21) Appl. No.: 18/853,726

(22) PCT Filed: Apr. 11, 2023
(86) PCT No.: PCT/EP2023/059360

§ 371 Date: Oct. 3, 2024

(30) Foreign Application Priority Data
Apr. 12, 2022 (EP) 22167985.5

(51) Int. Cl. C12N 15/113; C12N 15/86

(52) U.S. Cl. CPC C12N 15/113; C12N 15/86; C12N 2310/11; C12N 2310/531; C12N 2750/14143; C12N 2830/50

(57) ABSTRACT

The present invention relates to a nucleic acid comprising two or more RNA encoding sequences, wherein the each of the RNA comprises a hairpin and a guide sequence substantially complementary to part of a gene of choice, and to associated AAVs, compositions, pharmaceutical compositions and uses in treatments thereof.

### FIELD OF THE INVENTION

The present invention relates to the fields of biotechnology, medicine and gene therapy. Specifically, the invention relates to a nucleic acid comprising two or more RNA encoding sequences, wherein each of the RNA comprises a hairpin and a guide sequence substantially complementary to part of a gene of choice, and to associated AAVs, compositions, pharmaceutical compositions and uses in treatments thereof.

### BACKGROUND OF THE INVENTION

Hereditary diseases are caused by mutations in genes. According to the National Organization for Rare Disorders, there are several hundred million patients with hereditary diseases around the world, two-thirds of which are children. Currently, there are no effective therapies for more than 95% of these patients; furthermore, drug-based treatments approved for genetic diseases at best manage or modify symptoms. However, they do not address the underlying genetic cause of the disease and must be administered for life.

The goal of gene therapies for hereditary diseases aims at a one-time curative repair or change to an individual's affected gene that minimizes or even eliminates the symptoms for the entire life of the patient. One of the most promising strategies for human gene therapy is the use of adeno-associated virus (AAV) as a gene delivery vehicle, wherein the gene delivered by the vector does not integrate into the patient genome.

There are only a handful of gene therapy treatments currently approved for commercialization and readily available, including Luxturna (inherited retinal dystrophy), Zolgensma (spinal muscular atrophy), the two chimeric antigen receptor T cell (CAR-T) therapies (Yescarta and Kymriah), and Strimvelis (severe combined immunodeficiency due to adenosine deaminase deficiency (ADA-SCID).

Thus, there is a clear need for the development of new therapies. Specifically, within the AAV delivery field, there is a need for the development of highly effective and versatile systems that may target two or more transcripts with high efficiency.

### SUMMARY OF THE INVENTION

The present invention solves the problem in the prior art by using nucleic acids comprising at least two RNA encoding sequences, wherein each of the RNA comprises a hairpin and a guide sequence substantially complementary to part of a gene of choice. The invention provides for a highly versatile system, which allows for the simultaneous use of different guide sequences. As shown herein, the invention provides for an optimized and highly potent inhibition of RNA expression. Consequently, when using the nucleic acids or AAVs of the invention in gene therapy, a single-dose treatment is to be expected. This in turn results in lower toxicity and immunogenicity risks associated with the use of said nucleic acids or AAVs of the invention. Additionally, the costs of the therapy based on the nucleic acids or AAVs of the invention are expected to be lower than other gene therapies, presenting a substantial economic advantage.

Thus, a first aspect of the invention relates to a nucleic acid comprising a sequence encoding a first RNA and a sequence encoding a second RNA, wherein: the first RNA comprises a first guide sequence substantially complementary to a first target sequence in a first transcript encoded by a first human gene; the second RNA comprises a second guide sequence substantially complementary to a second target sequence in a second transcript encoded by a second human gene; the first RNA and the second RNA each comprise a hairpin; and the first RNA and the second RNA each comprise a sequence selected from the group consisting of: SEQ ID NO. 93; SEQ ID NO. 94; and variants thereof.

A second aspect of the invention relates to an expression cassette comprising the nucleic acid of the invention, where the expression cassette is a DNA molecule.

A third aspect of the invention relates to an adeno-associated virus (AAV) vector comprising the nucleic acid or expression cassette of the invention.

A fourth aspect of the invention relates to pharmaceutical compositions comprising the nucleic acid, expression cassette or the AAV vector of the invention.

A fifth aspect of the invention relates to the use as a medicament of the nucleic acid, the expression cassette, the AAV vector or the pharmaceutical composition of the invention.

A sixth aspect of the invention relate to a kit comprising the nucleic acid, the expression cassette, the AAV vector, or the pharmaceutical composition of the invention.

A seventh aspect of the invention relates to cells comprising the nucleic acid, the expression cassette, or the AAV vector of the invention.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may (in the case of a viral delivery vector) be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. 1990 J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Within the context of the present invention, the term “variant thereof”, when referring to a given sequence (SEQ ID), includes any nucleic acids which retain at least some of the properties of the corresponding native nucleic, for example, reduction of RNA expression. The term “variant” may include any nucleic acids with at least 50; 55; 60; 65; 70; 75; 80; 90; or 95% sequence identity with the native nucleic acid.

In some embodiments, the sequence of the nucleic acid of the invention is codon optimised. Codon optimisation refers to experimental approaches designed to improve the codon composition of a recombinant gene based on various criteria without altering the amino acid sequence. This is possible because most amino acids are encoded by more than one codon. Most codon-optimization approaches avoid the use of rare codons. However, different approaches vary in the extent of other features considered, including mRNA elements that can inhibit expression, nucleotide context of the initiation codon, mRNA secondary structures, sequence repeats, nucleotide composition, internal ribosome entry sites, promoter sequences, and putative splice donor and acceptor sites. In addition, some programs consider protein structural information, intragenic poly(A) sites, stop codons in alternative reading frames, and dinucleotides that are targets for RNase cleavage, mutation, and methylation-dependent gene silencing. The person skilled in the art has within their understanding the requirements needed to design such a codon-optimised nucleic acid.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6×sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly(A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA signal, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

### Detailed Description of the Invention

A first aspect of the invention relates to a nucleic acid comprising a first sequence encoding a first RNA and a second sequence encoding a second RNA, wherein: the first RNA comprises a first guide sequence substantially complementary to a first target sequence in a first transcript encoded by a first human gene; the second RNA comprises a second guide sequence substantially complementary to a second target sequence in a second transcript encoded by a second human gene; the first RNA and the second RNA each comprise a hairpin; and the first RNA and the second

RNA each comprise a sequence selected from the group consisting of: SEQ ID NO. 93; SEQ ID NO. 94; and variants thereof.

### Nucleic Acids

The term “nucleic acid” as used herein takes its regular meaning in the art. Thus, the term “RNA” or “RNA molecule” or “ribonucleic acid molecule” as used herein refers to a polymer of ribonucleotides (e.g., 2, 3, 4, 5, 10, 15, 20, 25, 30, or more ribonucleotides) and the term “DNA” or “DNA molecule” or “deoxyribonucleic acid molecule” as used herein refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. Preferably, a siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be induced, for example, to silence the expression of target genes. Double stranded RNA structures that are suitable for inducing RNAi are well known in the art. For example, a small interfering RNA (siRNA) can induce RNAi. An siRNA comprises two separate RNA strands, one strand comprising a first RNA sequence and the other strand comprising a second RNA sequence, thus a first and a second strand. An siRNA design that is often used involves consecutive base pairs with a 3′ overhang. The first and/or second strand may comprise a 3′-overhang. The 3′-overhang preferably is a dinucleotide overhang on both strands of the siRNA. Such a design is based on observed Dicer processing of larger double stranded RNAs that results in siRNAs having these features. The 3′-overhang may be comprised in the first strand. The 3′-overhang may be in addition to the first strand. The length of the two strands of which an siRNA is composed may be 19, 20, 21, 25 22, 23, 24, 25, 26 or 27 nucleotides or more.

siRNAs may also serve as Dicer substrates. For example, a Dicer substrate may be a 27-mer consisting of two strands of RNA that have 27 consecutive base pairs. The first strand is positioned at the 3′-end of the 27-mer duplex. At the 3′-ends, like with siRNAs, each or one of the strands may comprise a two nucleotide overhang. The 3′-overhang may be comprised in the first strand. The 3′-overhang may be in addition to the first strand. 5′ from the first strand, additional sequences may be included that are either complementary to the target RNA sequence adjacent or not. The other end of the siRNA Dicer substrate is blunt ended. This Dicer substrate design may result in a preference in processing by Dicer such that an siRNA can be formed like the siRNA design as described above, having 19 consecutive base pairs and 2 nucleotide overhangs at both 3′-ends.

In any case, siRNAs, or the like, are composed of two separate RNA strands (Fire et al. 1998, Nature. 1998 19; 391 (6669):806-11), each RNA strand comprising or consisting of the first and second RNA strand or the third and fourth RNA strand in accordance with the invention. Thus, the nucleic acid of the invention may be said to derive a first and second RNA strand, which is the first RNA, and a third and fourth RNA strand, which is the second RNA. Alternative naming conventions for each first, second, third and fourth RNA strands are within the scope of the invention, which may be complementary, substantially complimentary, or unique to each other, or in any other required arrangement as discussed herein.

The loop sequence may also be a stem-loop sequence, whereby the double stranded region of the shRNA is extended. Like the siRNA Dicer substrate described above, a shRNA can be processed by e.g. Dicer to provide for an siRNA having an siRNA design such as described above, having e.g. 19 consecutive base pairs and 2 nucleotide overhangs at both 3′-ends. In case the shRNA is to be processed by Dicer, it is preferred to have the first and second strands at the end of the shRNA, i.e. such that the putative strands of the siRNA are linked via a stem loop sequence: 5′-first strand—stem loop sequence—second strand—optional 2 nt overhang sequence—3′. Or, conversely, 5′-second strand—stem loop sequence—first strand—optional 2 nt overhang sequence—3′. Another shRNA design may be a shRNA structure that is processed by the RNAi machinery to provide for an activated RNA-induced silencing complex (RISC) that does not require Dicer processing (Liu et al. 2013 Nucleic Acids Res. 41(6):3723-33, incorporated herein by reference), so called AgoshRNAs, which are based on a structure very similar to the miR-451 scaffold as described below. Such a shRNA structure comprises in its loop sequence part of the first RNA sequence. Such a shRNA structure may also consist of the first strand, followed immediately by the second strand.

### Spacers

In preferred embodiments of the nucleic acid of the invention, the sequence encoding the first RNA is followed by a first spacer comprising at least 15 nucleotides and the sequence encoding the second RNA. Thus, preferably, in a 5′ to 3′ direction, the sequence encoding the first RNA is followed by a first spacer comprising at least 15 nucleotides, which spacer is followed by the sequence encoding the second RNA. It is understood that the 5′ to 3′ direction refers to the coding strand in case of a double-stranded (ds) nucleic acid.

As described above, the nucleic acid may be said to derive, in a 5′ to 3′ direction, a first and second RNA strand, which is the first RNA, and a third and fourth RNA strand, which is the second RNA, wherein the sequences encoding the first and second RNA strands are followed by a first spacer of at least 15 nucleotides followed by the sequences encoding the third and fourth RNA strands.

In some embodiments, the nucleic acid of the invention comprises a third sequence encoding a third RNA.

In preferred embodiments of the invention, the sequence encoding the third RNA is followed by a second spacer comprising at least 15 nucleotides followed by the sequence encoding the first RNA. Thus, preferably, in a 5′ to 3′ direction, the sequence encoding the third RNA is followed by a second spacer comprising at least 15 nucleotides, which second spacer is followed by the sequence encoding the first RNA, followed by the first spacer and the sequence encoding the second RNA.

Thus, the nucleic acid may be said to derive a first and second RNA strand, which is the first RNA, a third and fourth RNA strand, which is the second RNA, and a fifth and second RNA strands, which is the third RNA, wherein, in a 5′ to 3′ direction, the sequences encoding the fifth and sixth RNA strands are followed by a spacer of at least 15 nucleotides and the sequences encoding the first and second RNA strands followed by a spacer of at least 15 nucleotides and the sequences encoding the third and fourth RNA strands.

In some embodiments of the invention, the spacer comprises at least 25; at least 30; or at least 35 nucleotides.

In some embodiments of the invention, the spacer comprises at least: 15; 20; 25; 30; 35; 40; 45; 50; 55; 60; 65; 70; 75; 80; 85; 90; 95; 100; 105; 110; 115; 120; 125; 130; 135; 140; 145; 150; 155; 160; 165; 170; 175; 180; 185; 190; 195; or 200 nucleotides.

In some specific embodiments, the spacer of the invention comprises 15; 16; 17; 18; 19; 20; 21; 22; 23; 24; 25; 26; 27; 28; 29; 30; 31; 32; 33; 34; 35; 36; 37; 38; or 40 nucleotides.

In some specific embodiments of the invention, the spacer comprises 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; or 105 nucleotides.

In some embodiments of the invention, the spacer comprises or consists of a sequence selected from the group consisting of: SEQ ID NO: 46; SEQ ID NO. 47; SEQ ID NO. 48; and variants thereof.

In some embodiments of the invention, the sequences encoding the first and second RNA strands are followed by a spacer of at least 75 nucleotides and the sequences encoding the first and second RNA strands. In some specific examples of the invention, the sequences encoding the first and second RNA strands are followed by a spacer comprising or consisting of SEQ ID NO. 46.

In some embodiments of the invention, the sequences encoding the first and second RNA strands are followed by a spacer of at least 15 nucleotides and the sequences encoding the first and second RNA strands. In some specific examples of the invention, the sequences encoding the first and second RNA strands are followed by a spacer comprising or consisting of SEQ ID NO. 48.

In some embodiments of the invention, the sequences encoding the fifth and sixth RNA strands are followed by a spacer of at least 75 nucleotides and the sequences encoding the first and second RNA strands.

In some embodiments, the sequences encoding the fifth and sixth RNA strands are followed by a spacer of at least 75 nucleotides and the sequences encoding the first and second RNA strands are followed by a spacer of at least 15 nucleotides and the sequences encoding the third and fourth RNA strands. In some specific examples of the invention, the sequences encoding the fifth and sixth RNA strands are followed by a spacer comprising or consisting of SEQ ID NO. 46 and the sequences encoding the first and second RNA strands are followed by a spacer comprising or consisting of SEQ ID NO. 48 and the sequences encoding the third and fourth RNA strands.

In some embodiments of the invention, the first and second RNA comprise SEQ ID NO. 93; and the sequence encoding the first RNA is followed by a first spacer which comprises or consists of SEQ ID NO. 48.

In some embodiments of the invention, the first RNA comprises SEQ ID NO. 94 and the second RNA comprises SEQ ID NO. 93; and the sequence encoding the first RNA is followed by a first spacer which comprises or consists of SEQ ID NO. 46.

In some embodiments of the invention, the third RNA comprises SEQ ID NO. 54 and the first and second RNA comprise SEQ ID NO. 93; the sequence encoding the third RNA is followed by a second spacer which comprises or consists of SEQ ID NO. 46; and the sequence encoding the first RNA is followed by a first spacer which comprises or consists of SEQ ID NO. 48.

In some embodiments, if one or both RNAs are shRNA to be processed by Dicer, the relevant shRNA structures mentioned above are also applicable. In some embodiment, if one or both RNAs are AgoshRNAs, the relevant shRNA structures mentioned above are also applicable. Thus, one or both RNAs may be processed by the same or different RNAi machinery.

### RNA Scaffolds

A double stranded RNA according to the invention may be incorporated in a pre-miRNA or pri-miRNA scaffold. MicroRNAs, i.e. miRNA, are guide strands that originate from double stranded RNA molecules that are endogenously expressed e.g. in mammalian cells. A miRNA is processed from a pre-miRNA precursor molecule, similar to the processing of a shRNA or an extended siRNA as described above, by the RNAi machinery and incorporated in a RISC (Tijsterman M, Plasterk R H. Dicers at RISC; the mechanism of RNAi. Cell. 2004 Apr. 2; 1 17(1):1-3). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is a shRNA-like molecule that can subsequently be processed by Dicer to result in an siRNA-like double stranded RNA duplex. The miRNA, i.e. the guide strand, that is part of the double stranded RNA duplex is subsequently incorporated in RISC.

The first and second RNA of the invention comprise a first and a second guide sequence substantially complementary to a first and a second target sequence in (a first transcript encoded by) a first and a second gene. As further explained below, the first and second guide sequences may be the same sequence or different sequences. In some embodiments of the invention, the first and second genes targeted by the guide sequences comprised in the nucleic acid of the invention are viral genes. In some other embodiments of the invention, the first and second genes targeted by the guide sequences comprised in the nucleic acid of the invention are bacterial genes. In preferred embodiments of the invention, the first and second genes targeted by the guide sequences comprised in the nucleic acid of the invention are human genes.

An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31 (13), 3406-3415, 2003), the natural miRNA sequence as it is present in the RNA structure (i.e. duplex, pre-miRNA or primiRNA), and the sequence present in the structure that is substantially complementary therewith are removed and replaced with a first strand and a second strand according to the invention, that are the first strand and second strand of the first RNA, or the first strand and second strand of the second RNA, which may also be referred to as the third and fourth strands. Thus, using the first and second strand for solely exemplary purpose, the first strand and the second strand are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two separate RNA strands that are hybridized via complementary base pairing) as found in nature, are often not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first strand is described e.g. in Liu Y P Nucleic Acids Res. 2008 36(9):281 1-24.

A pri-miRNA can be processed by the RNAi machinery of the cell. The pri-miRNA comprising flanking sequences at the 5′-end and the 3′-end of a pre-miRNA hairpin and/or shRNA like molecule. Such a pri-miRNA hairpin can be processed by Drosha to produce a pre-miRNA. The length of the flanking sequences can vary but may be around 80 nt in length (Zeng and Cullen, J Biol Chem. 2005 280(30):27595-603; Cullen, Mol Cell. 2004 16(6):861-5). The minimal length of the single-stranded flanks can easily be determined as when it becomes too short, the RNA molecule may lose its function because e.g. Drosha processing fails resulting in sequence specific inhibition being reduced or even absent. In one embodiment, the pri-miRNA scaffold carrying the first and second strand according to the invention has a 5′-sequence flank and a 3′ sequence flank relative to the predicted pre-miRNA structure of at least 5, at least 10, at least 15, at least 20, at least 30, at least 40, or at least 50 nucleotides. Preferably, the pri-miRNA derived flanking sequences (5′ and 3′) comprised in the miRNA scaffold are derived from the same naturally occurring pri-miRNA sequence. Preferably, pre-miRNA and/or the pri-miRNA derived flanking sequences (5′ and 3′) and/or loop sequences comprised in the miRNA scaffold are derived from the same naturally occurring pri-miRNA sequence. As the (putative) guide strand RNA as comprised in the endogenous miRNA sequence can be replaced by a sequence including (or consisting of) the first strand, and the passenger strand sequence replaced by a sequence including (or consisting of) the second strand, it is understood that flanking sequences and/or loop sequences of the pri-miRNA or pre-miRNA sequences of the endogenous sequence may include minor sequence modifications such that the predicted structure of the scaffold miRNA sequence (e.g. M-fold predicted structure) is the same as the predicted structure of the endogenous miRNA sequence.

The first and second strands and the third and fourth strands, such as to form two double stranded RNAs, i.e. the first and second RNA of the invention, are encoded by an expression cassette. It is understood that, unless otherwise stated, any additional RNAs comprised in the nucleic acid of the invention, such as the third RNA of the invention as described below, are also encoded by the expression cassettes of the invention. It is also understood that when the double stranded RNAs are to be e.g. two siRNAs, consisting of two strands each, that there may be two or more expression cassettes required. When each double stranded RNA is comprised in a single RNA molecule, e.g. encoding a shRNA, pre-miRNA or pri-miRNA, one expression cassette per RNA molecule may suffice. A pol II expression cassette may comprise a promoter sequence a sequence encoding an RNA to be expressed followed by a polyadenylation sequence. In case the double stranded RNAs that are expressed comprise a pri-miRNA scaffold, the encoded RNA sequence may encode for intron sequences and exon sequences and 3′-UTR's and 5′-UTRs. A pol Ill expression cassette in general comprises a promoter sequence, followed by a sequence encoding an RNA (e.g. shRNA sequence, pre-miRNA, or a strand of the double stranded RNAs to be comprised in e.g. an siRNA or 5 extended siRNA). A pol I expression cassette may comprise a pol I promoter, followed by an RNA encoding sequence and a 3′-sequence. Expression cassettes for double stranded RNAs are well known in the art, and any type of expression cassette can suffice, e.g. one may use a pol III promoter, a pol II promoter or a pol I promoter (i.a. ter Brake et al., 2008 Mol Ther. March; 16(3):557-64, Maczuga et al., 2012 BMC Biotechnol. July 24; 12:42). In some embodiments, and as further described below, the expression cassette is a DNA molecule.

As is clear from the above, the first and second strands comprised in a double stranded RNA can contain additional nucleotides and/or nucleotide sequences. Any double stranded RNA of the invention may be comprised in a single RNA sequence or comprised in two separate RNA strands. Whatever design is used, it is designed such that, from the RNA sequence, an antisense or sense RNA molecule comprising the first or the second strand, as further explained below, in whole or a substantial part thereof, can be processed by the RNAi machinery, such that it is incorporated in the RISC complex to have its action, i.e. to induce RNAi against the RNA target sequence comprised in an RNA encoded by the gene of choice. The sequence comprising or consisting of the first or second strand, in whole or a substantial part thereof, is capable of sequence specifically targeting RNA encoded by the first and/or second gene of the invention. Hence, as long as the double stranded RNA is capable of inducing RNAi, such a double stranded RNA is contemplated in the invention.

In some embodiments, the double stranded RNAs according to the invention are comprised in a pre-miRNA scaffold, a pri-miRNA scaffold, a shRNA, or an siRNA. Preferably, the first and second strand of said double stranded RNA as encoded by the expressed cassette are to be contained in a single transcript. It is understood that the expressed transcript in subsequent processing, i.e. cleavage, results in the single transcript being processed into multiple separate RNA molecules.

The term complementary is herein defined herein as nucleotides of a nucleic acid sequence that can bind to another nucleic acid sequence through hydrogen bonds, i.e. nucleotides that are capable of base pairing. Ribonucleotides, the building blocks of RNA are composed of monomers (nucleotides) containing a sugar, phosphate and a base that is either a purine (guanine, adenine) or pyrimidine (uracil, cytosine). Complementary RNA strands form double stranded RNA. A double stranded RNA may be formed from two separate complementary RNA strands or the two complementary RNA strands may be comprised in one RNA strand. In complementary RNA strands, the nucleotides cytosine and guanine (C and G) can form a base pair, guanine and uracil (G and U), and uracil and adenine (U and A) can form a base pair as well. The term substantial complementarity means that is not required do have the first and second RNA sequence to be fully complementary, or to have the first RNA sequence and target RNA sequence or sequences of RNA encoded by a gene of choice to be fully complementary.

### miR-451

In some embodiments of the invention, the second RNA is incorporated in a pre-miRNA or a pri-miRNA scaffold derived from miR-451. In some embodiments of the invention, the second RNA comprises SEQ ID NO. 93 or a variant thereof.

In some other embodiments of the invention, each of the first and second RNA is incorporated in a pre-miRNA or a pri-miRNA scaffold derived from miR-451. In some specific embodiments of the invention, each of the first and second RNA comprises SEQ ID NO. 93 or a variant thereof.

Thus, and as shown in the examples, the first, second, third and fourth strands of the nucleic acid of the invention may be incorporated in a pre-miRNA or a pri-miRNA scaffold derived from miR-451. The miR-451 scaffold is found to be in particular useful within the present invention as it can induce RNA interference that can result in mainly guide strand induced RNA interference. The pri-miR-451 scaffold does not result in a passenger strand because the processing is different from the canonical miRNA processing pathway (Cheloufi et al. 2010 465(7298):584-9 and Yang et al., 2010 Proc Natl Acad Sci USA. 107(34):15163-8). The scaffolds represents an excellent candidate to develop a gene therapy product as unwanted potential off-targeting by passenger strands can be largely, if not completely, avoided. As the passenger strand (corresponding to the second sequence) may result in targeting of transcripts other than RNA encoded by the gene of choice, using such scaffolds may prevent such unwanted targeting. Hence, it is preferred that selected scaffolds produce less than 15%; less than 10%; less than 5%; less than 4%; or less than 3% of passenger strands.

A miR-451 scaffold, as shown in the examples, preferably comprises from 5′ to 3′, firstly 5′-CTTGGGAATGGCAAGG-3′ (SEQ ID NO. 92), followed by a sequence of 22 nucleotides, comprising or consisting of the first RNA sequence, followed a sequence of 17 nucleotides, which can be regarded as the second RNA sequence, which is complementary over its entire length with nucleotides 2-18 of said sequence of 22 nucleotides, subsequently followed by sequence 5′-MWCTTGCTATACCCAGA-3′ (wherein M is an A or a G or a C and W is an A or a U) (SEQ ID NO. 97). Preferably the first 5′-A/C/G nucleotide of the latter sequence is not to base pair with the first nucleotide of the first strand of the first or second RNA.

Such a scaffold may comprise further flanking sequences as found in the original pri-miR-451 scaffold. Alternatively, the hairpin stem sequences (SEQ ID NO. 92 and 97) may be replaced by hairpin stem sequences of other pri-mRNA structures. As is clear from the above, the sequence of the scaffold may differ not only with regard to the (putative) guide strand sequence, and sequence complementary thereto, as present in the wild-type scaffold, but may also comprise additional mutations in the 5′ sequence, loop and 3′ sequence as well, as additional mutations may be required to provide for an RNA structure that is predicted to mimic the secondary structure of the wild-type scaffold. Such a scaffold may be comprised in a larger RNA transcript, e.g. a pol II expressed transcript, comprising e.g. a 5′ UTR and a 3′UTR and a poly A. Flanking structures may also be absent.

In some preferred embodiments, the first strand of the first RNA and the first strand of the second RNA have a length of 19, 20, 21, or 22 nucleotides. In some specific embodiments, the first strand of the first RNA and the first strand of the second RNA are fully complementary over its entire length with the target sequence. In some preferred embodiments, the first strand of the first RNA and the first strand of the second RNA have a length of 19, 20, 21, or 22 nucleotides, wherein each said first strand of the first RNA and first strand of the second RNA are fully complementary over its entire length with the target sequence.

As described herein, the first strand of the first RNA and the first strand of the second RNA are to be combined with a second strand of the first RNA and a second strand of the second RNA, respectively. The skilled person is well capable of designing and selecting a suitable second strand of the first and second RNA in order to provide for a first and second strand for the first and second RNA that can induce RNA interference when expressed in a cell.

### miR-144

Said third RNA may be incorporated in a pre-miRNA or a pri-miRNA scaffold derived from miR-144. In some specific embodiments of the invention, the third RNA comprises SEQ ID NO. 54 or a variant thereof. In other words, and as described above, the first and second strands of the third RNA of the invention are incorporated in a pre-miRNA or a pri-miRNA scaffold derived from miR-144. Thus, the third RNA can be described as a hairpin or a double stranded RNA that is substantially complementary to itself. In some embodiments, the hairpin in the second RNA comprises at least 70 nucleotides.

In some specific embodiments, the nucleic acid of the invention comprises a first, a second and a third RNA, wherein the first and second RNA are incorporated in a pre-miRNA or a pri-miRNA scaffold derived from miR-451 and the third RNA is incorporated in a pre-miRNA or a pri-miRNA scaffold derived from miR-144. In some specific embodiments, the first and second RNA comprises SEQ ID NO. 93 or a variant thereof and the third RNA comprises SEQ ID NO. 54 or a variant thereof. The variants thereof comprise SEQ ID NO. 36.

In said embodiments where the third RNA is incorporated in a pre-miRNA or a pri-miRNA scaffold derived from miR-144, the third RNA is processed by Dicer; therefore, the putative strands of the subsequent siRNA are linked via a stem loop sequence: 5′-first strand—apical loop sequence—second strand—optional 2 nt overhang sequence—3′ or, conversely, 5′-second strand—apical loop sequence—first strand—optional 2 nt overhang sequence—3′.

In some embodiments of the invention where the third RNA is incorporated in a pre-miRNA or a pri-miRNA scaffold derived from miR-144, the third RNA is mutated to reduce processing and/or expression of the third RNA. In some specific embodiments, SEQ ID NO. 54 or the variant thereof is mutated to reduce processing and/or expression of the third RNA.

In some embodiments, the mutation is a single point mutation. In other words, the third RNA comprises a single point mutation to reduce processing and/or expression of the third RNA. Any mismatch, bulge or GU wobble introduced within positions 4-8 of the DROSHA cleavage site may impair the enzymatic activity of DROSHA. Double and triple mismatches, bulges or wobbles within said positions further decrease the activity of DROSHA. Therefore, any of the following single nucleotide polymorphisms (SNPs) and combinations thereof within the 4-8 nucleotide stretch of miR-144 may alter (pre-)-miR-144 expression.

In some embodiments, the third RNA comprises at least one mutation selected from the group consisting of: U>G at position 4; A>U or G at position 5; U>A at position 6; C>G or U at position 7;and A>U or G at position 8.

In preferred embodiments, the third RNA comprises: a single point mutation A>U at position 5. The skilled person can easily determine whether this is the case by using standard assays and appropriate controls such as described in the examples and as known in the art.

The specific combination of miR-144/miR-451 scaffolds, specifically those comprising SEQ ID NO. 54 or SEQ ID NO. 36 and those comprising SEQ ID NO. 93, within the nucleic acid of the invention is particularly helpful within the context of gene therapy and RNA silencing. miR-144 and miR-451 are examples of clustered miRNAs regulated in trans, wherein miR-144 regulates the processing of miR-451 by Ago2. Specifically, miR-144 enhances miR-451 biogenesis in trans by repressing Dicer and, in turn, repressing global canonical miRNA processing (Kretov et al., 2020, Molecular Cell 78, 317-328). Thus, where this combination of scaffolds is used, the third RNA of the invention plays a key role in enhancing the biogenesis of the first and second RNA of the invention, and therefore, the delivery of the guide sequences comprised in said first and second RNAs.

In some embodiments of the invention, the first RNA is incorporated into a miR-144 scaffold.

In some specific embodiments of the invention, the first RNA is incorporated into a miR-144 scaffold, and the second RNA is incorporated into a miR-451 scaffold.

As stated above, the first RNA comprises a first guide sequence. In those embodiments of the invention where the first RNA is incorporated into a miR-144 scaffold, the first guide sequence may be incorporated into the first or the second strand of the first RNA.

In those embodiments where the first RNA of the invention is incorporated into a miR-144 scaffold, the miRNA 144 scaffold for use in the invention preferably comprises from 5′ to 3′, firstly 5′-TGGGGCCCTGGCTM-3′ (wherein M is an A or a C or a G or a U) (SEQ ID NO. 95), followed by a sequence of 22 nucleotides, comprising or consisting of a first RNA sequence, followed by a sequence of 15 nucleotides which can be regarded as the apical loop 5′-TTTGCGATGAGAWMM-3′ (wherein W is preferably a C or a G, but can also be a A or a U, and is to base pair with the last nucleotide of the first strand of the first RNA; and wherein M is an A or a C and is not to base pair with nucleotide 21 of the first strand of the first RNA) (SEQ ID NO. 96), followed by a sequence of 20 nucleotides, which can be regarded as the second RNA sequence, and which is complementary over its entire length with nucleotides 1 and 3-10 and 12-20 of said sequence of 22 nucleotides, except nucleotide 18 that forms a mismatch with nucleotide 2 of the first strand, subsequently followed by sequence 5′-AGTCCGGGCACCCCC-3′ (SEQ ID NO. 98).

Such a scaffold may comprise flanking sequences as found in the original pri-miR-144 scaffold. Alternatively, the flanking sequences 5′-ATCGGCGCTATGCTTCCTGTGCCCCCAG-3′ (SEQ ID NO. 44) and 5′-AGCTCTGGAG CCTGACAAGGAGGACAGGAGAGATGCTGCAAGCCCAAGAAGCTCTCTGCTCAGCCTGTCAC AACCTACTGACTGCCAGGGCA-3′ (SEQ ID NO. 46) may be replaced by flanking sequences of other pri-mRNA structures.

In those embodiments where the first RNA of the invention comprises a miR-144 scaffold, the guide sequence may be comprised within the 5p- or 3p-arm of the scaffold. DROSHA processing of miR-144, subsequently followed by DICER processing, generate a miRNA duplex which ultimately enters the process of miRNA strand selection. Strand selection is operated within the RISC and determines which strand will become the active strand (also referred to as the guide strand) and which strand will be degraded (passenger strand) (Noland and Doudna, 2013 RNA, 19:639-648). Guide strand selection is highly determined by thermodynamic characteristics of the miRNA duplex, and thus it may be influenced by modifying the nucleotide sequence encoding the miRNA. Generally speaking, the strand with the lower thermodynamic stability at its 5′ end will become the guide strand. Another key feature of human miRNA guide strands is a 5′ end U-bias associated with an enrichment for A and G nucleotides, whilst the passenger strands exhibit a 5′ end C-bias and an enrichment for C and U nucleotides (Hai Yang Hu et al. 2009, BMC Genomics 2009, 10:413).

Conveniently, since the guide strand selection is sequence dependent, the miR-144 constructs of the invention comprising guide strands may be designed as 5p- or 3p-guide-containing regions (SEQ ID NO 95). Importantly, the probability with which the desired guide strand of the invention will be selected in RISC can be modulated by single nucleotide variations such as selecting U as the first nucleotide of the first strand (5p-design) or the first nucleotide of the second strand (3p-designs) and selecting C or G as nucleotide 20 of the first strand (5p-designs) or nucleotide 19 of the second strand (3p-designs).

As with miR-451 scaffolds, the sequence of the miR-144 scaffold may differ not only with regard to the (putative) guide strand sequence, and sequence complementary thereto, as present in the wild-type scaffold, but may also comprise additional mutations in the 5′, loop and 3′ sequence as well, as additional mutations may be required to provide for an RNA structure that is predicted to mimic the secondary structure of the wild-type scaffold. Again, such a scaffold may be comprised in a larger RNA transcript, e.g. a pol Il expressed transcript, comprising e.g. a 5′ UTR and a 3′UTR and a poly A. Flanking structures may also be absent. An expression cassette in accordance with the invention may thus express a shRNA-like structure having a sequence of 22 nucleotides, comprising or consisting of the first strand of the first RNA, followed a sequence of 20 nucleotides, which can be regarded to be the second strand of the first RNA, which is complementary over its entire length with nucleotides 1 and 3-10 and 11-19 of said sequence of 22 nucleotides, except nucleotide 18 that forms a mismatch with nucleotide 2 of the first strand. The latter shRNA-like structure derived from the miR-144 scaffold can be referred to as a pre-miRNA scaffold from miR-144. Alternatively, the flanking sequences, 5′-ATCGGCGCTATGCTTCCTGTGCCCCCAG-3′ and 5′-AGCTCTGGAGCCTGACAAGGAGGACAGGAGAGATGCTGCAAGCCCAAGAAGCTCTCTGCTC AGCCTGTCACAACCTACTGACTGCCAGGGCA-3′ may be replaced by flanking sequences of other pri-mRNA structures. Flanking structures may also be absent.

In some preferred embodiments, the first or the second strand of the first RNA has a length of 19, 20, 21, or 22 nucleotides. In some specific embodiments, the first or second strand of the first RNA is fully complementary over its entire length with the first target sequence. In some preferred embodiments, the first or the second strand of the first RNA has a length of 19, 20, 21, or 22 nucleotides, wherein said first strand of the first RNA is fully complementary over its entire length with the target sequence.

As explained above, where the first strand of the first RNA comprises a guide sequence, the first strand is to be combined with a second strand of the first RNA. As above, the skilled person is well capable of designing and selecting a suitable second strand of the first RNA.

Similarly, where the second strand of the first RNA comprises a guide sequence, the second strand is to be combined with a first strand of the first RNA. As above, the skilled person is well capable of designing and selecting a suitable first strand of the first RNA

A suitable scaffold comprising a first and second strand for the first RNA in accordance with the invention can be a sequence such as SEQ ID NO. 94.

Thus, in some embodiments of the invention, the first RNA comprises SEQ ID NO. 94 or a variant thereof. In some more specific embodiments of the invention, the first RNA comprises SEQ ID NO. 94 or a variant thereof; and the second RNA comprises SEQ ID NO. 93 or a variant thereof.

Hence, where the first RNA comprises SEQ ID NO. 94, the first guide sequence may be incorporated into the first or the second strand of the first RNA.

As shown in the examples, modifying the miR-144 scaffold does not affect its helper function on miR-144; the expression of guide sequences from both 3p and 5p miR-144 leads to an optimal target lowering, and, further, modulation of the guide/passenger ratio can be achieved to enrich for the desired strand, providing for a novel and efficient miRNA expression system.

### Guide Strands

The first and second RNA to be expressed in accordance with the invention comprise, in whole or a substantial part thereof, a guide strand.

In certain embodiments of the invention, the guide strand may also be referred to as antisense strand as it is complementary (“anti”) to a target RNA sequence in a sense or antisense transcript, the sense or antisense target RNA sequence being comprised in an RNA encoded by a gene of choice. Thus, the first and second RNA also comprise a “sense strand”, that may have substantial sequence identity with, or be identical to, the target RNA sequence. Therefore, the first and second RNA can be described as a hairpin or a double stranded RNA that is substantially complementary to itself.

Said double stranded RNA according to the invention is to induce RNA interference, thereby reducing expression of transcripts encoded by the gene of choice, which includes knocking down of said transcripts. Transcripts that may be targeted may include spliced, including splice variants, and unspliced RNA transcripts. Thus, an RNA encoded by a gene of choice is understood to comprise unspliced mRNAs comprising a 5′ untranslated region (UTR), intron and exon sequences, followed by a 3′ UTR and a polyA signal, and also splice variants thereof. The double stranded RNA according to the invention may also induce transcriptional silencing.

Reducing expression of a transcript encoded by the gene of choice is herein thus preferably understood as reducing the steady state level of a functional mRNA encoded by the gene of choice in a target cell such that less of the mRNA is available in the cell for translation into the product encoded by the gene of choice, thereby reducing the steady state level of the protein in the target cell. Reducing expression of a transcript encoded by the gene of choice therefore does not necessarily involve reducing de novo transcription of the gene but rather increased degradation of an mRNA encoded by the gene of choice and/or its precursors, e.g. unspliced RNA transcripts.

In some embodiments, the double stranded RNA according to the invention comprises a first RNA sequence and a second RNA sequence, wherein the first and second RNA sequences are substantially complementary, and wherein the first RNA sequence has a sequence length of at least 19 nucleotides and is substantially complementary to a target sequence in a transcript encoded by a first or second gene, which first RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce the first and second gene expression.

Similarly, in some embodiments, the double stranded RNA according to the invention comprises a first RNA sequence and a second RNA sequence, wherein the first and second RNA sequences are substantially complementary, and wherein the second RNA sequence has a sequence length of at least 19 nucleotides and is substantially complementary to a target sequence in a transcript encoded by a first or second gene, which first RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce the first and second gene expression.

The skilled person can easily determine whether this is the case by using standard luciferase reporter assays and appropriate controls such as described in the examples and as known in the art (Zhuang et al. 2006 Methods Mol Biol. 342:181-7). For example, a luciferase reporter comprising a target RNA sequence can be used to show that the double stranded RNA according to the invention is capable of sequence specific knock down. Furthermore, levels of gene expression can be determined by detecting endogenous mRNA levels (nuclear and/or cytoplasmic), RNA foci formation, and endogenous protein levels.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

It is understood that “substantially complementary” in this context means that it is not required to have all the nucleotides of the guide sequence and the target sequence to be base paired, i.e. to be fully complementary, or all the nucleotides of the guide sequence and the target sequence to be base paired. As long as the first and second RNAs of the invention are capable of inducing RNA interference to thereby sequence-specifically target a sequence comprising the target RNA sequence, such substantial complementarity is contemplated in accordance with the invention.

The substantial complementarity between the strand complimentary to the target RNA sequence, which may also be referred to as part of the first or second gene, preferably consists of at most two mismatched nucleotides, more preferably having one mismatched nucleotide, most preferably having no mismatches. It is understood that one mismatched nucleotide means that over the entire length of the strand complimentary to the target RNA sequence when base paired with the target RNA sequence one nucleotide does not base pair with the target RNA sequence. Having no mismatches means that all nucleotides of the strand complimentary to the target RNA base pair with the target RNA sequence, having 2 mismatches means two nucleotides of the strand complimentary to the target RNA do not base pair with the target RNA sequence. The strand complimentary to the target RNA may also comprise additional nucleotides that do not have complementarity to the target RNA sequence, and may be longer than e.g. 21 nucleotides. In such a scenario, the substantial complementarity is determined over the entire length of the target RNA sequence. This means that the target RNA sequence in this embodiment has either no, one or two mismatches over its entire length when base paired with the strand complimentary to the target RNA.

As shown in the example section, double stranded RNAs comprising a strand complimentary to different targets were tested. These strands complimentary to the target RNA had no mismatches and were fully complementary with the target RNA sequence. Having a few mismatches between the strand complimentary to the target RNA and the target RNA sequence may however be allowed according to the invention, as long as the double stranded RNA according to the invention is capable of reducing expression of transcripts comprising the target RNA sequence, such as a luciferase reporter or e.g. a transcript comprising the target RNA sequence. In said embodiments, substantial complementarity between the strand complimentary to the target RNA and the target RNA sequence consists of having no, one or two mismatches over the entire length of either the strand complimentary to the target RNA or the target RNA sequence encoded by an RNA of the first or second human gene, whichever is the shortest.

As said, a mismatch according to the invention means that a nucleotide of the first (or second) and third strand (the strand) does not base pair with the target RNA sequence encoded by an RNA of the first or second gene. Nucleotides that do not base pair are A and A, G and G, C and C, U and U, A and C, C and U, or A and G. A mismatch may also result from a deletion of a nucleotide, or an insertion of a nucleotide. When the mismatch is a deletion in the strand sequence, this means that a nucleotide of the target RNA sequence is not base paired with the sequence when compared with the entire length of the strand sequence. Nucleotides that can base pair are A-U, G-C and G-U. A G-U base pair is also referred to as a G-U wobble, or wobble base pair. In one embodiment the number of G-U base pairs between the strand sequence and the target RNA sequence is 0, 1 or 2. In some embodiments, there are no mismatches between the strand RNA sequence and the target RNA sequence and a G-U base pair or G-U pairs are allowed. Preferably, there may be no G-U base pairs between the strand sequence and the target RNA sequence, or the strand sequence and the target RNA sequence only have base pairs that are A-U or G-C. Preferably, there are no G-U base pairs and no mismatches between the strand sequence and the target RNA sequence. The strand sequence of the double stranded RNA according to invention preferably is fully complementary to the target RNA sequence, said complementarity consisting of G-U, G-C and A-U base pairs. The strand sequence of the double stranded RNA according to invention more preferably is fully complementary to the target RNA sequence, said complementarity consisting of G-C and A-U base pairs.

Therefore, in some embodiments, the strand sequence and the target RNA sequence have at least 15, 16, 17, 18, or 19 nucleotides that base pair. Preferably the strand and the target RNA sequence are substantially complementary, said complementarity comprising at least 19 base pairs. In other embodiments, the strand has at least 8, 9, 10, 11, 12, 13 or 14 consecutive nucleotides that base pair with consecutive nucleotides of the target RNA sequence. In other embodiments, the strand has at least 19 consecutive nucleotides that base pair with consecutive nucleotides of the target RNA sequence. In other embodiments, the strand comprises at least 19 consecutive nucleotides that base pair with 19 consecutive nucleotides of the target RNA sequence. In other embodiments, the strand has at least 17 nucleotides that base pair with the target RNA sequence and have at least 15 consecutive nucleotides that base pair with consecutive nucleotides of the target RNA sequence. The sequence length of the first strand is preferably at most 21, 22, 23, 24, 25, 26, or 27 nucleotides. In other embodiments, the strand has at least 20 consecutive nucleotides that base pair with 20 consecutive nucleotides of the target RNA sequence. In other embodiments, the strand comprises at least 21 consecutive nucleotides that base pair with 21 consecutive nucleotides of the target RNA sequence.

As said, it may be not required to have full complementarity (i.e. full base pairing (no mismatches) and no G-U base pairs) between the first or second strand of the first RNA and the first strand of the second RNA, and the target RNA sequence as such a strand can still allow for sufficient suppression of gene expression. Also, not having full complementarity may be contemplated for example to avoid or reduce off-target RNA sequence specific gene suppression while maintaining sequence specific inhibition of transcripts comprising the target RNA sequence. However, it may be preferred to have full complementarity as it may result in more potent inhibition. Without being bound by theory, having full complementarity between the first or second strand of the first RNA and the first strand of the second RNA, and the target RNA sequence may allow for the activated RISC complex comprising said first or second strand of the first RNA or the first strand of the second RNA (or a substantial part thereof) to cleave its target RNA sequence, whereas having mismatches may hamper cleavage and can result in mainly allowing inhibition of translation, of which the latter may result in less potent inhibition.

With regard to the second strand on the RNA of the invention, the second strand is substantially complementary with the first strand on the RNA. The second strand combined with the first strand forms a double stranded RNA. As said, this is to form a suitable substrate for the RNA interference machinery such that a guide sequence derived from the first strand is comprised in the RISC complex in order to sequence specifically inhibit expression of its target, i.e. RNA encoded by the first or second gene. The sequence of the second strand has sequence similarity with the target RNA sequence. However, the substantial complementarity of the second strand with the first strand may be selected to have less substantial complementarity as compared with the substantial complementarity between the first strand and the target RNA sequence. Hence, the second strand may comprise 0, 1, 2, 3, 4, or more mismatches, 0, 1, 2, 3, or more GU wobble base pairs, and may comprise insertions of 0, 1, 2, 3, 4, nucleotides and/or deletions of 0, 1, 2, 3, 4, nucleotides. It is understood that, where the guide sequence is comprised within the second strand of the first RNA, or the second strand of the invention, the description above applies to the first strand of the invention.

Preferably the first strand and the second strand are substantially complementary, said complementarity comprising 0, 1, 2 or 3 G U base pairs and/or wherein said complementarity comprises at least 17 base pairs. These mismatches, G-U wobble base pairs, insertions and deletions, are with regard to the first strand, i.e. the double stranded region that is formed between the first and second strands. As long as the first and second strands can substantially base pair, and are capable of inducing sequence specific inhibition of an RNA encoded by the first or second gene, such substantial complementarity is allowed according to the invention. It is also understood that substantially complementarity between the first and the second strands may depend on the double stranded RNA design of choice. It may depend for example on the miRNA scaffold that is chosen for in which the double stranded RNA is to be incorporated.

As clear from the above, the substantial complementarity between the first strand and second strand of the first and second RNA may comprise mismatches, deletions and/or insertions relative to a first and second RNA sequence being fully complementary (i.e. fully base paired). In some embodiments, the first and second strands of the first and/or second RNA have at least 11 consecutive base pairs. Hence, at least 11 consecutive nucleotides of the first strand and at least 11 consecutive nucleotides of the second strand are fully complementary. In some embodiment the first and second strands of the first and/or second RNA have at least 15 nucleotides that base pair. Said base pairing between at least 15 nucleotides of the first strand and at least 15 nucleotides of the second strand may consist of G-U, G-C and A-U base pairs, or may consist of G-C and A-U base pairs.

In some embodiments, the first and second RNA sequences have at least 15 nucleotides that base pair and have at least 11 consecutive base pairs. In another embodiments, the first RNA sequence and the second RNA sequence are substantially complementary, wherein said complementarity comprises at least 17 base pairs. Said 17 base pairs may preferably be 17 consecutive base pairs, said base pairing consisting of G-U, G-C and A-U base pairs or consisting of G-C and A-U base pairs.

The invention thus provides for an expression cassette encoding the first strand and second strand of the first and second RNA wherein the first and second strands are substantially complementary, wherein the first or second strand of the first RNA and the first strand of the second RNA has a sequence length of at least 19 nucleotides and is substantially complementary to a target RNA sequence comprised in an RNA encoded by the first or second gene, respectively gene.

In some embodiments, an expression cassette is provided encoding a first strand and a second strand wherein the first and second strands of the first and second RNA are substantially complementary, wherein the first strand or second strand of the first RNA and the first strand of the second RNA has a sequence length of at least 22 nucleotides and is substantially complementary to a target RNA sequence comprised in an RNA encoded by the first or second gene. Where applicable, the expression cassette may also encode the first strand and second strand of the third RNA, wherein the first and second strands are substantially complementary.

As stated above, the RNAs of the invention may be incorporated into miRNA scaffolds. The miRNA scaffold sequence is processed by the RNAi machinery as present in the cell. In some specific embodiments of the invention, the processing of the miRNA scaffold sequence results in: guide sequences comprising the first strand of the first RNA, or a substantial part thereof, in the range of 21-30 nucleotides; and guide sequences comprising the first strand of the second RNA, or a substantial part thereof, in the range of 21-30 nucleotides. In some other specific embodiments of the invention, the processing of the miRNA scaffold sequence results in: guide sequences comprising the second strand of the first RNA, or a substantial part thereof, in the range of 21-30 nucleotides; and guide sequences comprising the first strand of the second RNA, or a substantial part thereof, in the range of 21-30 nucleotides.

Such guide strands are capable of reducing the first and second gene transcript expression by targeting the selected target sequences.

As shown in the examples, the first strand of the first RNA and the first strand of the second RNA, or, alternatively, the second strand of the first RNA and the first strand of the second RNA, as it is encoded by the expression cassette of the invention, is comprised in part or in whole, in a guide strand when it has been processed by the RNAi machinery of the cell. Hence, the guide strand that is to be generated from the RNA encoded by the expression cassette, comprising the first or second strand of the first RNA and the first strand of the second RNA, is to comprise at least 18 nucleotides of the second RNA sequence.

Preferably, such a guide strand comprises at least 19 nucleotides, 20 nucleotides, 21 nucleotides, or 22 nucleotides. A guide strand can comprise the first or second strand of the first or the first strand of the second RNA sequence, also as a whole. In selecting a miRNA scaffold, the first or second strand of the first RNA sequence and the first strand in the second RNA sequence, can be selected such that it is to replace the original guide strand. However, this does not necessarily mean that the guide strand produced from such an artificial scaffold is identical in length to the first or second strand of the first RNA or the first strand of the second RNA selected, nor that the first or second strand of the first RNA or the first strand of the second RNA is in its entirety to be found in the guide strand that is produced.

In some embodiments of the nucleic acid of the invention, the guide sequences are selected from the group consisting of SEQ ID NO. 1 to SEQ ID NO. 6. In some embodiments, the complementary strand sequences to SEQ ID NO. 1 to 6 are SEQ ID NO. 14 to SEQ ID NO. 19, respectively. In some specific embodiments of the nucleic acid of the invention, the guide sequence comprised within the 5p-arm of SEQ ID NO. 94 is SEQ ID NO. 9. In some embodiments, the complementary strand sequence to SEQ ID NO. 9 is SEQ ID NO. 22.

In some embodiments of the nucleic acid of the invention, the guide sequence comprised within the 3p-arm of SEQ ID NO. 94 is selected from the group consisting of SEQ ID NO. 10 to SEQ ID NO. 12. In some embodiments, the complementary strand sequences to SEQ ID NO. 10 to 12 are SEQ ID NO. 23 to SEQ ID NO. 25, respectively.

In certain embodiments, the first and second RNA of the invention are selected from the group consisting of SEQ ID NO. 28 to 33; and SEQ ID NO. 37 to SEQ ID NO. 40.

In certain embodiments, the 5′-flanking region of the first or third RNA of the invention is SEQ ID NO. 42. In certain embodiments, the 5′-flanking region of the first or third RNA of the invention is SEQ ID NO. 43. In certain embodiments, the 5′-flanking region of the first or third RNA of the invention is SEQ ID NO. 44. In some embodiments, the 3′-flanking region of the second RNA of the invention is SEQ ID NO. 45.

In some embodiments of the invention, there is provided an expression cassette selected from the group consisting of SEQ ID NO. 55 to SEQ ID NO. 81.

In some embodiments of the invention, the cassette is operably linked to a promoter comprising or consisting of SEQ ID NO. 50. In some embodiments, the cassette is linked to a poly A signal comprising or consisting of SEQ ID NO. 51.

In some embodiments of the invention, the first RNA comprises SEQ ID NO. 93; the second RNA comprises SEQ ID NO. 94; and the third RNA comprises SEQ ID NO. 54.

In some embodiments of the invention, the first RNA comprises SEQ ID NO. 94; the second RNA comprises SEQ ID NO. 93; and the third RNA comprises SEQ ID NO. 54.

In a second aspect of the invention, there is provided an expression cassette comprising the nucleic acid of the invention, wherein the expression cassette is a DNA molecule. In some specific embodiments, the nucleic acid comprised within the cassette is operably linked to a promoter and optionally to a poly-A signal. In some embodiments, the expression cassette comprises, in 5′ to 3′ order, the following elements: at least one promoter, at least a first and second RNA and at least a poly A signal.

In some embodiments, there is provided the expression cassette as disclosed herein, further comprising a nucleic acid encoding at least one gene. In a further embodiment, the gene is encoded by a coding portion (e.g. cDNA) of a naturally occurring gene. In some embodiments, the gene product is a protein, or fragment thereof. In a further embodiment, the gene does not comprise a sequence that is substantially complementary to any one or more of the guide sequences as defined herein. In a further embodiment, the gene comprises a nucleotide sequence that is codon optimised for expression in human cells. In a further embodiment, the gene is codon optimised to differ sufficiently from the endogenous gene sequence in cells such that it would not be recognised by any shRNAs targeting a wild-type version of the gene. The person skilled in the art has within their understanding the requirements needed to design such a nucleic acid.

The nucleotide sequence or expression cassettes as defined above for expression in a mammalian cell comprises at least one mammalian cell-compatible expression control sequence, e.g. a promoter, that is/are operably linked to the sequence coding for the gene product of interest, thus forming an expression cassette for expression of the gene product of interest in mammalian target cell to be treated by gene therapy with the gene product of interest. Many such promoters are known in the art (see Sambrook and Russel, 2001, supra). Constitutive promoters that are broadly expressed in many cell-types, such as the CMV promoter, may be used. However, more preferred will be promoters that are inducible, tissue-specific, cell-type-specific, or cell cycle-specific. Preferably a pol Il promoter is used, such as a CAG promoter (i.a. Miyazaki et al. 1989 Gene 79 (2): 269-77; Niwa N. et al. 1991 Gene. 108 (2): 193-9), a PGK promoter, or a CMV promoter (Such as depicted e.g. in FIG. 2 of WO2016102664). For any diseases primarily affect the brain, it may be particularly useful to use a neurospecific promoter.

Thus, in some embodiments of the invention, the promoter is a promoter capable of driving transcription in a brain cell. Examples of suitable neurospecific promoters are Neuron-Specific Enolase (NSE), native or engineered chicken beta-actin (CAG) promoter, human synapsin 1, caMK kinase and tubuline (Hioki et al. 2007 Gene Ther. 14(11):872-82). Other suitable promoters that can be contemplated are inducible promoters, i.e. a promoter that initiates transcription only when the host cell is exposed to some particular stimulus.

The expression cassette comprising the nucleic acid of the invention encodes a polyA signal. In some embodiments of the invention, the polyA signal is the simian virus 40 polyadenylation (SV40 polyA); or Bovine Growth Hormone polyadenylation (BGH polyA) or Human Growth Hormone polyadenylation signal (HGH polyA).

The expression cassettes according to the invention can be transferred to a cell, using e.g. transfection methods. Any suitable means may suffice to transfer an expression cassette according to the invention. Preferably, the expression cassettes according to the invention are comprised in a viral vector, preferably a gene therapy vector. Preferably, gene therapy vectors are used that stably transfer the expression cassette to the cells such that stable expression of the double stranded RNAs that induce sequence specific inhibition of the first and second gene can be achieved. Suitable vectors may be lentiviral vectors, retrotransposon based vector systems, or AAV vectors. It is understood that as e.g. lentiviral vectors carry an RNA genome, the RNA genome will encode for the said expression cassette such that after transduction of a cell, the said DNA sequence and said expression cassette is formed. Preferably, the gene therapy vector is a viral vector. Preferably the viral vector is an AAV. Therefore, in some embodiments, an expression cassette as disclosed herein is flanked by Inverted Terminal Repeats.

Thus, in preferred embodiments of the invention, the expression cassette comprising the nucleic acid of the invention is flanked by at least one AAV Inverted Terminal Repeat (ITR). In some specific embodiments, the expression cassette is flanked by one 5′ ITR and one 3′ ITR. In other words, the expression cassette is flanked by an ITR sequence at the 5′ end of the cassette and an ITR sequence at the 3′ end of the cassette.

### AAVs

A third aspect of the present invention relates to an adeno-associated virus (AAV) comprising the expression cassette of the invention.

In some embodiments of the invention, the AAV comprises the expression cassette of the invention.

Recombinant parvoviruses, in particular dependoviruses such as infectious human or simian adeno-associated virus (AAV), and the components thereof (e.g. a parvovirus genome), may be used as vectors for introduction and/or expression of nucleic acids in mammalian cells, preferably human cells. An AAV vector is defined as a recombinantly produced AAV or AAV particle that comprises a polynucleotide to be delivered into a host cell, either in vivo, ex vivo or in vitro. Herein, an AAV vector construct refers to the polynucleotide comprising the viral genome or part thereof, usually at least one ITR, and a transgene. Herein, a transgene refers to a nucleotide sequence of interest and may comprise promotor and/or regulatory sequences necessary for expression as well as sequences encoding the gene of interest.

A “recombinant parvoviral or AAV vector” (or “rAAV vector”) or a “parvoviral or AAV vector” herein refers to a parvoviral or AAV virion (i.e. a capsid), comprising (or “packaging”) one or more nucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such (r)AAV vectors can be replicated and packaged into infectious viral particles when present in a suitable host cell that is expressing AAV rep and cap gene products (i.e. AAV Rep and Cap proteins). When the transgene(s) of interest that is/are flanked by at least one ITR is incorporated into a larger nucleic acid construct (e.g. in a chromosome or in another vector such as a plasmid or baculovirus used for cloning or transfection), this is typically referred to as a “pro-vector” which can be “rescued” by replication and encapsidation in the presence of AAV packaging functions and necessary helper functions.

Preferably the AAV vector that is used is an AAV vector of serotype 5 or serotype 9. AAVs of serotype 5 or 9 (also referred to as AAV5 or AAV9) may be in particularly useful for transducing human neurons and human astrocytes such as shown in the examples. Therefore, in some embodiment, there is an AAV comprising an expression cassette as disclosed herein. Thus, AAV5 and AAV9 can efficiently transduce different human cell types of the CNS including FBN, dopaminergic neurons, motor neurons and astrocytes and is therefore a suitable vector candidate to deliver therapeutic genes to the CNS to treat neurogenerative diseases, including but not limited to the treatment of Alzheimer's Disease via targeting e.g. the first and second gene as described herein. The production of AAV vectors comprising any expression cassette of interest is well described in; WO2007/046703, WO2007/148971, WO2009/014445, WO2009/104964, WO2011/122950, WO2013/036118, which are incorporated herein in its entirety.

AAV sequences that may be used in the present invention for the production of AAV vectors, e.g. produced in insect or mammalian cell lines, can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71:6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. AAV capsids may consist of VP1, VP2 and VP3, but may also consist of VP1 and VP3.

In some embodiments, the AAV vector according to the inventions comprises an AAV5 or AAV9 capsid proteins. In some embodiment, the AAV vector according to the inventions comprises a AAV5 capsid protein. In some embodiment, the AAV vector according to the inventions comprises a AAV9 capsid protein.

It is to be understood that the capsid amino acid sequences and the nucleotide sequences encoding them can be engineered, for example, the sequence may be a hybrid form or may be codon optimized, such as for example by codon usage. The capsid proteins may be engineered, for example, via DNA shuffling, error prone PCR, bioinformatics rational design or site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, CA, USA; Sep. 1-5, 2004, pages 3520-3523; Asuri et al., 2012, Molecular Therapy 20(2):329-3389; Lisowski et al., 2014, Nature 506(7488):382-386, herein incorporated by reference.

In some embodiments, the ITRs and capsid proteins (or parts thereof) in the AAV vector of the invention may be from different AAV serotypes. By way of example, and not limitation, the ITRs may be derived from AAV2, whilst the capsid proteins may be derived from a different serotype, for example AAV5 or AAV9.

In another embodiment, a host cell is provided comprising the said nucleic acid or said expression cassette according to the invention. For example, the said expression cassette or nucleic acid may be comprised in a plasmid contained in bacteria. Said expression cassette or nucleic acid may also be comprised in a production cell that produces e.g. a viral vector. Said expression cassette may also be provided in a baculovirus vector.

Various modifications of the nucleotide sequences as defined above, including e.g. the wildtype AAV sequences, for proper expression in the host cell is achieved by application of well-known genetic engineering techniques such as described e.g. in Sambrook and Russell (2001, supra). Various further modifications of coding regions are known to the skilled artisan which could increase yield of the encode proteins. These modifications are within the scope of the present invention.

In one embodiment, any mammalian cell may be infected by an AAV vector of the invention, for example, but not limited to, a muscle cell, a liver cell, a nerve cell, a glial cell and an epithelial cell and the mammalian species may be any species including, but not limited to, murine, dog, non-human primate and human. In a preferred embodiment the mammalian cell to be infected is a human cell.

In a fourth aspect, the present invention relates to a pharmaceutical composition (“the pharmaceutical composition of the invention”) comprising the nucleic acid of the invention, the expression cassette of the invention, or the AAV vector of the invention, and at least one pharmaceutically acceptable excipient.

Suitable excipients include, but are not limited to, buffers and stabilizers, antioxidants, etc. By way of example, the pharmaceutical composition of the invention may comprise physiological buffers, such as e.g. PBS, and stabilizing agents such as e.g. sucrose.

The compositions of the invention are compatible with and suitable and intended for use in subsequent intravenous, intrathecal, intrastriatal, intracerebellar, intraparenchymal, intravitreal, subretinal administration or for use in organ-targeted vascular delivery such as intraporal or intracoronary delivery or isolated limb perfusion.

In some embodiments the pharmaceutical composition of the invention may also comprise at least one immunosuppressive compound. Said compound may reduce and/or prevent an immune response induced by administration of the pharmaceutical composition of the invention.

In some embodiments these compositions are used to transduce cells in vitro or ex vivo, in which case the excipients will need to be compatible with cell culture.

In a fourth aspect, the invention relates to the nucleic acid of the invention, the expression cassette of the invention, the AAV vector of the invention, or the pharmaceutical composition of the invention for use as a medicament. The appropriate definitions are provided elsewhere in this application.

In some embodiments, said medicament reduces expression of RNA encoded by the first and second gene, as explained herein above.

In some embodiments, the first and the second gene are the same.

In some specific embodiments, the first and the second target sequence are the same. In said embodiments, the potency of the medicament is maximized when compared to equivalent medicaments comprising AAVs bearing only one RNA sequence targeting the said same sequence.

Within the context of this invention, “potency” is understood as an expression of the activity of a medicament or therapeutic substance, in terms of the concentration or amount needed to produce a defined effect. The potency of a given medicament may be expressed as the concentration (EC50) or dose (ED50) of the drug required to produce 50% of that medicament's maximal effect. In certain cases, relative potency may be used, where instead of using units to describe the dose required to achieve a certain endpoint, a ratio of equivalent doses is used. By way of example, medicament A is 10 times more potent than medicament B i.e., it achieves the same effect with 1/10^{th} of the dose.

In some specific embodiments, the first target sequence is different from the second target sequence.

Where the first and second gene are the same, the potency of the medicament is again maximized when compared to equivalent medicaments comprising AAVs comprising only one RNA sequence. By way of non-limiting example, the first and second target sequence may reduce expression of transcripts encoded by variants of the gene.

Where the first target sequence is different from the second target sequence, the first gene may also be different from the second gene. In certain embodiments, the first and second genes encode a first and a second polypeptides, respectively, which are present within the same metabolic pathway. In other embodiments, the first and second genes encode a first and a second polypeptides which are present within a first and a second metabolic pathway, respectively.

Within the context of the present invention, a metabolic pathway can be defined as a set of actions or interactions between genes and their products that results in the formation or change of some component of the system, essential for the correct functioning of a biological system. In particular, within the context of the invention, the metabolic pathways are generally associated with the development of a certain disease. In some embodiments, the first and second metabolic pathways are associated with the development of the same disease. In other embodiments, the first metabolic pathway is associated with the development of a first disease, and the second metabolic pathway is associated with the development of a second disease.

The diseases that can be treated using the nucleic acid or the AAV of the invention are not particularly limited, other than by generally having a genetic cause or basis. The diseases that may be treated with the disclosed vectors include, but are not limited to, Parkinson's disease (PD), Lewy body Dementia (LBD), multiple system atrophy, Amyotrophic lateral sclerosis (ALS), frontotemporal dementia (FTD), atypical presentations mimicking neurodegenerative brain diseases, acute intermittent porphyria (AIP), age-related macular degeneration, Alzheimer's disease, arthritis, Batten disease, Canavan disease, Citrullinemia type 1, Crigler Najjar, congestive heart failure, cystic fibrosis, Duchene muscular dystrophy, dyslipidemia, glycogen storage disease type I (GSD-I), hemophilia A, hemophilia B, hereditary emphysema, homozygous familial hypercholesterolemia (HoFH), Huntington's disease (HD), Leber's congenital amaurosis, methylmalonic academia, ornithine transcarbamylase deficiency (OTC), PD, phenylketonuria (PKU), spinal muscular atrophy, paralysis, Wilson disease, epilepsy, focal cortical dysplasia (FCD), Pompe disease, Tay-Sachs disease, hyperoxaluria 9PH-1), spinocerebellar ataxia type 1 (SCA-1), SCA-3, u-dystrophin, Gaucher's types II or III, arrhythmogenic right ventricular cardiomyopathy (ARVC), Fabry disease, familial Mediterranean fever (FMF), proprionic acidemia, fragile X syndrome, Rett syndrome, Niemann-Pick disease and Krabbe disease. Examples of therapeutic gene products to be expressed include C9ORF72, SNCA, N-acetylglucosaminidase, alpha (NaGLU), Treg167, Treg289, EPO, IGF, IFN, GDNF, FOXP3, Factor VIII, Factor IX and insulin.

As used herein, the term “treat” and any of its variants refers to any kind of healthcare which is intended to relieve or eliminate the symptoms and/or causes of illness, injury, mental health problems, etc. The term “prevent” and any of its variants refers to any action taken to decrease the chance of getting a disease or condition.

It is understood that treatment of said diseases may be provided not only to human subjects suffering from any of said diseases, but also to human subjects having a genetic predisposition of developing the diseases who may or may not show symptoms of the disease.

The methods for producing the nucleic acid of the invention comprise any methods for producing nucleic acids, including but not limited to de novo synthesis, all of which would be apparent to the skilled person.

The method for producing the AAV vector of the invention may comprise the steps of: a) culturing a host cell as herein defined above under conditions such that the AAV vector is produced; and, b) optionally, one or more of recovery, purification and formulation of the AAV vector.

The host cell is a host cell that is suitable for the production of AAV vectors. Accordingly, the host cell is a host cell that is amenable to in vitro culture, preferably at large scale. Host cells that are suitable for the production of AAV vectors are well-known in the art and will typically be a mammalian or an insect cell line. Mammalian cell lines for producing AAV vectors are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell. Mammalian cell lines for producing AAV vectors in particular include a broad range of HEK293 cell lines, of which the HEK293T cell line is preferred.

Insect cell lines for producing AAV vectors can be any insect cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

Thus, in some embodiments, the expression cassette or construct is an insect cell-compatible vector or a mammalian cell-compatible vector. An “mammalian cell-compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, or infection.

The AAV in the supernatant can be recovered and/or purified using suitable techniques which are known to those of skill in the art. For example, monolith columns (e.g., in ion exchange, affinity or IMAC mode), chromatography (e.g., capture chromatography, fixed method chromatography, and expanded bed chromatography), centrifugation, filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques. In addition, recovery may preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In general, suitable methods for producing an AAV vector according to the invention in mammalian or insect host cells, and means therefore (such as expression constructs for expression of AAV rep proteins), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798.

The methods for producing the pharmaceutical composition of the invention comprise any methods for producing pharmaceutical compositions, all of which would be apparent to the skilled person. Said methods generally comprise the combination of the nucleic acid or the AAV of the invention with excipients under specific conditions.

Another aspect of the invention relates to a kit comprising the nucleic acid of the invention, the expression cassette of the invention, the AAV vector of the invention, or the pharmaceutical composition of the invention, and an immunosuppressive compound.

In certain embodiments of the invention, the immunosuppressive compound may reduce and/or prevent an immune response induced by administration of the nucleic acid, the AAV vector, or the pharmaceutical composition of the invention.

In another aspect, the invention relates to a cell comprising the nucleic acid of the invention or the AAV of the invention, or a host cell.

In some embodiments, the cell of the invention is a prokaryote cell. In some specific embodiments, the cell of the invention is a bacterial cell. In some embodiments, the cell of the invention is a eukaryote cell. In some embodiments, the cell of the invention is a mammalian cell. In some embodiments, the cell of the invention is an insect cell.

The nucleic acid of the invention, the expression cassette of the invention, or the AAV vector of the invention may be delivered into the cell of the invention by any suitable methods, included but not limited to, transfection, transformation, transduction, nucleofection, electroporation, and microinjection. For example, the expression cassette or nucleic acid of the invention may be comprised in a plasmid contained in bacteria. The expression cassette or nucleic acid of the invention may also be comprised in a production cell that produces e.g. a viral vector. Said expression cassette may also be provided in a baculovirus vector.

Further detail on host cells comprising an AAV vector according to the invention has been provide elsewhere in this application. Any mammalian cell may be infected by the AAV vector of the invention, including but not limited to a muscle cell, a liver cell, a nerve cell, a glial cell or an epithelial cell. In some preferred embodiments of the invention, the cell to be infected is a human cell.

In another aspect, the present invention relates to a method of treating or preventing a disorder, wherein the method comprises administering the nucleic acid of the invention, the expression cassette of the invention, or the AAV of the invention to a subject, thereby treating or preventing the disorder.

In another aspect, the invention relates to the nucleic acid of the invention, the expression cassette of the invention, or the AAV of the invention for use in the manufacture of a medicament for treatment of a disorder.

### BRIEF DESCRIPTION OF THE FIGURES

FIG. 1. Validation of the miR-144-miR-451 cluster scaffold by small RNA sequencing and luciferase reporter lowering. A) Schematic representation of single hairpin scaffold miR-451 and clustered miR-144-miR-451 scaffold. B) Comparison of mature microRNA expression from miR-451, expression of mature miR-144, and mature miR-16 (housekeeping miRNA) across scaffolds containing pri-miR-451 or pri-miR-144 wild type (WT)-pri-miR-451 cluster. Quantification was done by small RNA sequencing on human HEK293T transfected cells. Data are presented as mean values +/−SD. The asterisks (****p<0.0001) indicate statistical significant differences from two-tailed unpaired t-test. C) Schematic representation of clustered miR-144 SeedM-mutant-miR-451 scaffold. D) Comparison of mature miR-144, mature miRNA1 expressed from miR-451 and miR-16 (housekeeping miRNA) expression from various seed modified miR-144-miR-451 scaffolds. Quantification was done by small RNA sequencing on human HEK293T transfected cells. Data are presented as mean values +/−SD. The asterisks (*p=0.0364; *p=0.0326) indicate statistical significant differences from the unpaired t-test. E) Assessment of knock-down efficiency of seed modified miR-144-miR-451 scaffolds expressing miRNA1. HEK293T cells were co-transfected with the scaffolds (0.05 pg pDNA/cell) and a miRNA1 targeted luciferase reporter (1:2 pDNA/reporter ratio). Data are presented as mean values +/−SD. F) Comparison of expression values of a panel of synthetic miRNA sequences (miRNA1, miRNA2, miRNA3 and miRNA4) inserted within miR-451 of miR-144 A>TmiR-451 scaffold. Quantification was done by small RNA sequencing on human HEK293T transfected cells. Data are presented as mean values +/−SD. The asterisks (****p<0.0001; ***p=0.0008; **p=0.0046; *p=0.0215) indicate statistical significant differences from the two-tailed unpaired t-test.

FIG. 2. Schematic representation of the miR-451 and miR-144-miR-451 scaffolds and the first and second generations of scaffolds of the invention (Scaffolds B, C, D and E; respectively).

FIG. 3. Detailed representation of a cassette of the invention, including the promoter, Scaffold B of the invention (as defined below), the polyadenylation sequence, the spacer and flanking regions.

FIG. 4. Validation of scaffolds A, B and C of the invention. A) Schematic representation of scaffolds of the invention (Scaffolds A, B and C). B) Comparison of miRNA1, miRNA5 and miR-144 A>T expression values across the scaffolds by small RNA sequencing. Data are presented as mean values +/−SD. The asterisks (****p=0.0001) indicate statistical significant differences from the two-tailed unpaired t-test.

FIG. 5. Validation of scaffolds A and D of the invention. A) Schematic representation of scaffolds of the invention (Scaffolds A and D). B) Comparison of miRNA3 and miRNA4 expression values across the scaffolds by small RNA sequencing. Data are presented as mean values +/−SD. The asterisks (**p=0.052; *p=0.0262) indicate statistical significant differences from the two-tailed unpaired t-test.

FIG. 6. Validation of scaffolds A and E of the invention. A) Schematic representation of scaffolds of the invention (Scaffolds A and E). B) Comparison of miRNA1 and miRNA2 expression values across the scaffolds by small RNA sequencing. Data are presented as mean values +/−SD. The asterisks (****p<0.0001; ***p=0.0007; **p=0.097) indicate statistical significant differences from the two-tailed unpaired t-test.

FIG. 7. Assessment of knock-down efficiency of scaffolds A, B and C of the invention expressing miRNA1 and miRNA5 by dual luciferase assay. A) HEK293T cells were co-transfected with the different scaffolds (0.5 pg pDNA/cell) and a miRNA1 targeted luciferase reporter (5:1 pDNA/reporter ratio). B) HEK293T cells co-transfected with the different scaffolds (0.5 pg pDNA/cell) and a miRNA5 targeted luciferase reporter (5:1 ratio).

FIG. 8. Assessment of knock-down efficiency of scaffolds A, B and C of the invention expressing miRNA1 and miRNA5 at endogenous mRNA level. A) HEK293T cells were transfected with the different scaffolds (1 pg pDNA/cell) and miRNA1 targeted transcript expression was assessed by RT-qPCR. B) HEK293T cells were transfected with the different scaffolds (1 pg pDNA/cell) and miRNA5 targeted transcript expression was assessed by RT-qPCR.

FIG. 9. In vitro validation of the scaffold version 1 as a mean for single target lowering using rAAV. A) Schematic representation of scaffolds of the invention (Scaffolds A1, A2 and B3) B) HEK293T cells were transduced with a MOI of 1×10E+07 of rAAV9 at either 1:1 ratio of rAAV9 expressing scaffold A1 and A2 or full ratio of rAAV9 expressing scaffold B3. C) Assessment of transduction efficiency (vDNA) by qPCR. D) Assessment of miRNA1 and miRNA2 expression by RTqPCR. Data are presented as mean values +/−SD. The asterisks (*p=0.0141) indicate statistical significant differences from the two-tailed unpaired t-test. E) Assessment of target knock-down efficiency by RTqPCR. (LLOD=Lower limit of detection; LLOQ=Lower limit of quantification). Data are presented as mean values +/−SD. The asterisks (*p=0.0293) indicate statistical significant differences from the two-tailed unpaired t-test.

FIG. 10. A) Detailed representation of a cassette of the invention, including the promoter, scaffold F of the invention, the polyadenylation sequence, the spacer and flanking regions. B) Schematic representation of Scaffold F1 detailing the synthetic miRNA1 and miRNA5 integration sites. C) Sequence and folding of human endogenous miR-144 and its derivative scaffolds, F1 5p, F1 3p, F1 3p19C, and F1 3p1G. The integrated miRNA guide sequence is highlighted in bold, introduced mutant nucleotides are shown underlined. D) Most left panel shows guide and passenger strand expression assessed by small RNA sequencing for scaffolds F1 of the invention. Data are plotted as the guide/passenger (G/P) ratio. Right panel shows the expression of mature miRNA1 over miR-16 (housekeeping microRNA) across samples transfected with the scaffolds F1. HEK293T cells were transfected with 1 pg pDNA/cell. Data are presented as mean values +/−SD. The asterisks (**p=0.0057; ****p<0.0001) indicate statistical significant differences from the two-tailed unpaired t-test. E) Analysis of the isomiR expression of miRNA5 and miRNA1 across samples transfected with the scaffolds F1 5p, F1 3p, F1 3p19C and F1 3p1G. F) Assessment of knock-down efficiency of scaffolds F1 5p, F1 3p, F1 3p19C and F1 3p1G expressing miRNA1 and miRNA5 by dual luciferase assay (5 to 1 pDNA:reporter ratio). HEK293T cells were co-transfected with the different scaffolds and a miRNA1 or miRNA5 targeted luciferase reporter (5 to 1 pDNA:reporter ratio). G) Assessment of knock-down efficiency of scaffolds F1 of the invention and its variants expressing miRNA1 and miRNA5 at endogenous mRNA level. HEK293T cells were transfected with the different scaffolds (1 pg pDNA/cell) and miRNA1 targeted transcript expression as well as miRNA5 targeted transcript expression were assessed by RT-qPCR.

FIG. 11. Validation of the miR-144-miR-451 cluster scaffold version 2 in vitro and in vivo. A) Schematic representation of Scaffold F2 detailing the synthetic miRNA integration sites (miRNA6 within miR-144-5p and miRNA7 within miR-451). B) Left graph shows the expression of mature miRNA6 guide and passenger strands and mature miRNA7 in transfected HEK293T cells with scaffold F2 (0.5 pg pDNA/cell) as assessed by small RNA sequencing. Right graph represents guide (miRNA6)/passenger (miRNA6*) ratio (G/P). C) Analysis of the isomiR expression of miRNA6 and miRNA7 in samples transfected with the scaffold F2. D) Assessment of knock-down efficiency of scaffold F2 expressing miRNA6 and miRNA7 by dual luciferase assay (6 to 1 pDNA: reporter ratio). HEK293T cells were co-transfected with the scaffold F2 and a miRNA6 or miRNA7 targeted luciferase reporter. Data are presented as mean values +/−SD. The asterisks (**p=0.0028; ****P<0.0001) indicate statistical significant differences from the two-tailed unpaired t-test. E) Scaffold F2 was packaged into an AAV5 vector and injected in the striatum of mice. F) Assessment of transduction efficiency (vDNA) in the striatum of rAAV5-[Scaffold F2] intra-cerebral injected mice. G) Assessment of miRNA6 and miRNA7 expression (left graph) and guide/passenger ratio (right graph). H) Assessment of miRNA6 and miRNA7 processing by small RNA sequencing in mice striatum after rAAV5-[Scaffold F2] injection. I) Quantification of 5′ exact seed processing for miRNA6 and miRNA7 guide strands in mice striatum.

### EXAMPLES

### Materials and Methods

### Expression Cassettes: Designs and Cloning

miRNA1, miRNA2, miRNA3, miRNA4, miRNA5, miRNA6, miRNA7 and the scrambled controls, guide strand (miRNA SEQ ID NO. 1 to SEQ ID NO. 6 and SEQ ID NO. 9 to 12 and SEQ ID NO. 100 to 101; scrambled SEQ ID NO. 7, 8 and 13) and complementary strand (miRNA SEQ ID NO. 14 to SEQ ID NO. 19; SEQ ID NO. 22 to 25; and SEQ ID NO. 20, 21, 26, 102 and 103 (scrambled) were embedded into: the human pri-miR-451 sequence (SEQ ID NO. 27), resulting in SEQ ID NO. 28 to 35 and 104; and/or the human pri-miR-144 sequence (SEQ ID NO. 36), resulting in SEQ ID NO. 37 to 41 and 105. The miR-451 scaffold was flanked by a 157 nucleotides 5′ flanking region (SEQ ID NO. 42), whereas the pri-miR-451 and pri-miR-144 part of scaffolds A to F were flanked by a 28 nucleotides 5′ flanking region (SEQ ID NO. 43 (A-D and F) and 44 (E), respectively). All miR-451 and scaffolds A to F were flanked by a 205 nucleotides 3′ flanking region (SEQ ID NO. 45), followed by a BamHI restriction site and a 269 bp or 133 bp or 101 bp polyadenylation signal (SEQ ID NO. 51, 108 and 109). For the avoidance of doubt, when referring to scaffolds A to F of the invention, the term “scaffold” refers to the combination of RNAs of the invention, comprised within different hairpins or miR-144 and/or miR-451 scaffolds.

Each association of hairpins comprises a spacer sequence that separates a first hairpin and a second hairpin. miR-144 and miR-451 are separated by a endogenous 92 bp spacer sequence (SEQ ID NO. 46); miR-451 and miR-144 are separated by the reversed 92 bp spacer sequence (SEQ ID NO. 47); and miR-451 and miR-451 are separated by a synthetic 30 bp spacer sequence (SEQ ID NO. 48).

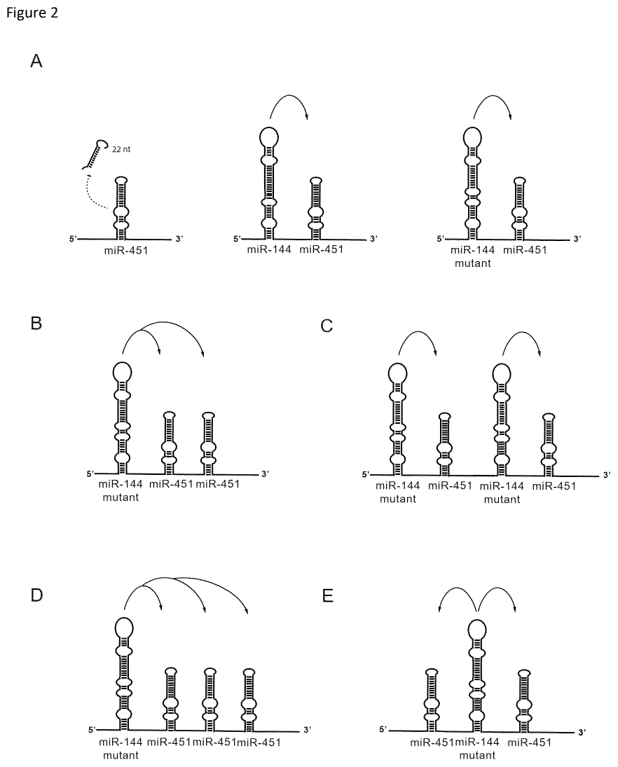

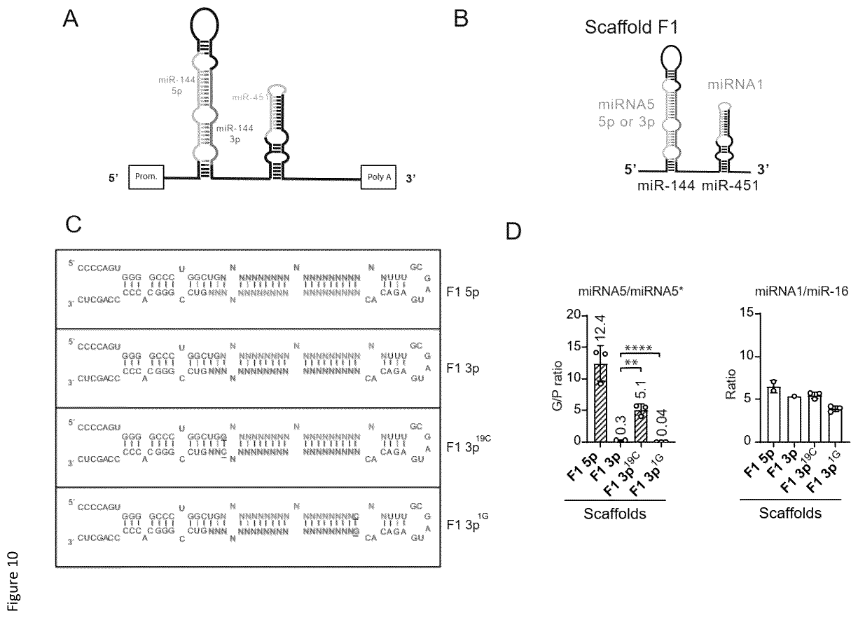

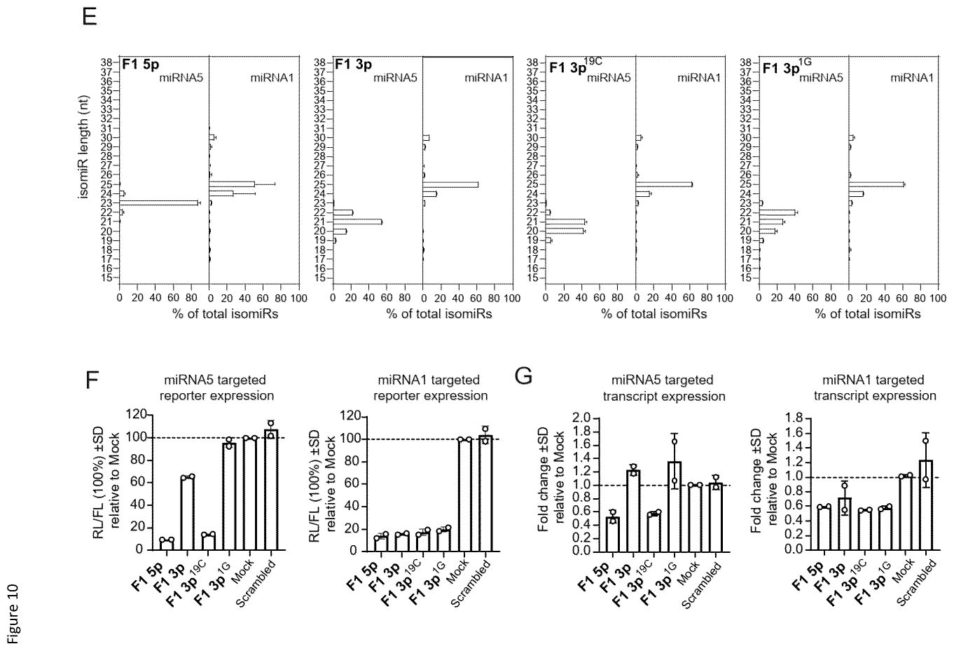

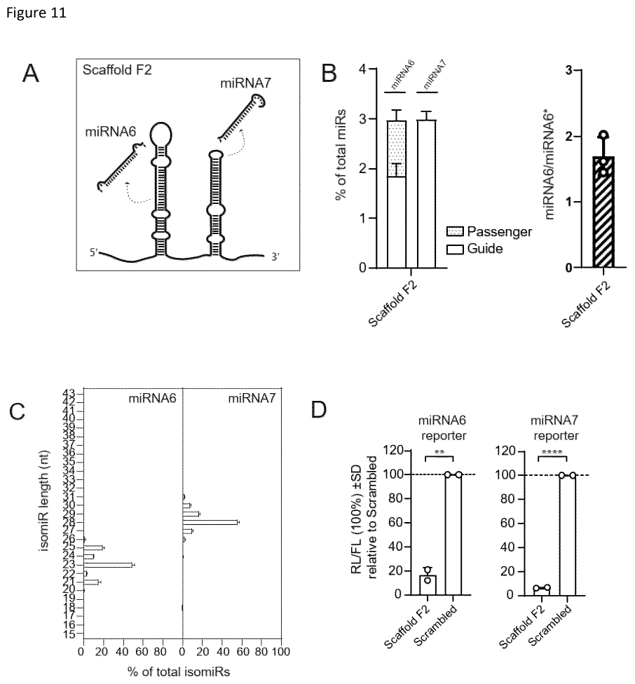

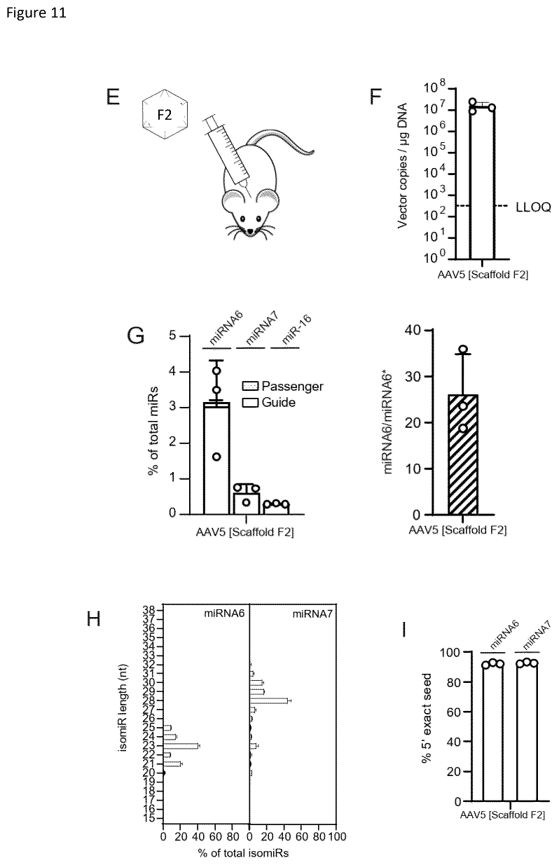

NheI and NotI restriction sites were added at the 5′-and 3′-ends, respectively, and the complete sequence was synthesized and subcloned into a pUC screening vector (SEQ ID NO. 49) (GeneWiz, Azenta Life Sciences). The cassettes comprising scaffolds A to E of the invention (SEQ ID NO. 55-81) were expressed from the CMV immediate-early enhancer fused to chicken β-actin promoter (CAG promoter, SEQ ID NO. 50) and terminated by the bovine growth hormone polyadenylation (bgh polyA, SEQ ID NO. 51) signal (FIG. 2). The cassettes comprising scaffolds F1 and its derivatives (FIG. 10) of the invention (SEQ ID NO. 77 to 81) were expressed from the CMV immediate-early enhancer fused to chicken β-actin promoter (CAG promoter, SEQ ID NO. 50) and terminated by the bovine growth hormone polyadenylation (bgh polyA, SEQ ID NO. 51) signal (FIG. 10). The cassettes comprising scaffolds F2 of the invention and its derivatives (FIG. 11) (SEQ ID NO. 106) were expressed from the hybrid form of the CBA promoter (Alt promoter, SEQ ID NO. 107) and terminated by the simian virus 40 polyadenylation (SV40 polyA, SEQ ID NO.108) signal (FIG. 10).

### DNA Constructs for Baculovirus Seed Generation

The expression cassettes A1, A2 and B3 (SEQ ID NO. 62, 63 and 125) were incorporated in a plasmid encoding the AAV ITRs. The cassettes comprising scaffolds A1, A2 and B3 of the invention were expressed from the hybrid form of the CBA promoter (Alt promoter, SEQ ID NO. 107) and terminated by the human growth hormone polyadenylation signal (hgh polyA, SEQ ID NO. 109). The ITR plasmid subcloning was performed by GeneWiz (Azenta Life Sciences). An example of a representative viral vector sequence is listed in SEQ ID NO.110, which comprises the Alt promoter—Cassette A1—hgh polyA.

### AAV5 Vectors

The expression cassette F2 (SEQ ID NO.106) was incorporated in a plasmid encoding the AAV ITRs. The cassette comprising scaffold F2 of the invention was expressed from the hybrid form of the CBA promoter (Alt promoter, SEQ ID NO. 107) and terminated by the simian virus 40 polyadenylation (SV40 polyA, SEQ ID NO. 108) signal. The ITR plasmid subcloning was performed by GeneWiz (Azenta Life Sciences). An example of a representative viral vector sequence is listed in SEQ ID NO.124, which comprises the Alt promoter—Cassette F2—SV40 polyA. Recombinant AAV5 were produced by PEI transfection of HEK293T cells with two plasmids encoding for Rep-Cap and the ITR-Cassette F2 containing transgene plasmid (Sirion Biotech). Following two step purification with primary capture with POROS™ CaptureSelect™ AAV-X resin (Thermo Fisher Scientific) and iodixanol gradient the titer of the purified AAV was determined using QPCR.

### Animal Studies

To investigate the in vivo expression efficacy of [miR-144(miRNA6)5p-miR-451(miRNA7)], AAV5 vectors were generated encoding Scaffold F2 as described above. A cohort of transgenic BAC-C9-112 female and male mice were injected in the striatum with doses of 3E+10-1.5E+11 gc/hemisphere. After the infusion, the mice were followed up until 20 weeks (12 weeks post-infusion) of age with weekly body weight measurements and cage side observations. At 20 weeks of age, the animals are euthanized and the following tissue samples were collected and fresh-frozen: cortex (frontal, caudal), hippocampus, striatum, thalamus, brainstem, cerebellum, hypothalamus, ventral midbrain, spinal cord C, T and L segments. Snap frozen samples were used to extract DNA for vector genome quantification and RNA for miRNA6 and miRNA7 quantification by small RNA sequencing. DNA and RNA were isolated using the AllPrep DNA/RNA 96-well kit (QIAGEN) following manufacturer's instructions. Vector genome copies were quantified by using TaqMan QPCR assay (Thermo Fisher scientific) (SEQ ID NO. Primer/probe SV40 111 and 112 and 113) and qualified standard lines. The QPCR was performed on 7500 Fast Real-Time PCR System (Thermo Fisher Scientific) and analysed on 7500 Software (v2.3) (Thermo Fisher Scientific).

### Culture of HEK293T Cells

The HEK293T cells were maintained in Dulbecco's modified Eagle's medium (Thermo Fisher Scientific) containing of heat inactivated fetal bovine serum (Thermo Fisher Scientific), at 37° C. and 5% CO2. For transfection and transduction assays, cells were seeded in 96 or 24-well plates at a density of 2E+04 or 1E+05 cells per well respectively one day prior transfection or transduction.

### AAV9 Transduction of HEK293T Cells and Molecular Analysis

Cells were transduced with AAV9 vectors at a multiplicity of infection (MOI) of 1E+07 total genome copies (gc) per cell in duplicate. Two days post-transduction, cells were harvested in 350 μl RTL plus buffer (AllPrep DNA/RNA Micro Kit, Qiagen) and genomic DNA and total RNA were isolated for subsequent molecular analysis.

### Vector DNA Isolation and Quantification of Vector Copies

DNA extraction was performed using AllPrep DNA/RNA Micro Kit (Qiagen) and following manufacturer's instructions. Vector genome copies were quantified by using TaqMan QPCR assay (Thermo Fisher scientific) (SEQ ID NO 114 and 115 and 116) and qualified standard lines. The QPCR was performed on 7500 Fast Real-Time PCR System (Thermo Fisher Scientific) and analysed on 7500 Software (v2.3) (Thermo Fisher Scientific).

### RNA Isolation and Quantification of Micro RNA Copies

Total RNA extraction was performed using AllPrep DNA/RNA Micro Kit (Qiagen) following the manufacturer's instructions. To quantify the expression of synthetic miRNAs, micro-RNA specific assays were developed. In brief, in order to detect miRNA1 and miRNA2 expression levels, isolated RNA was reverse transcribed into cDNA by using a TaqMan® MicroRNA Reverse Transcription Kit (Applied Biosystems, 4366597) and a gene specific RT primer to target miRNA1 or miRNA2 (relevant SEQ ID NO. 117 and 118). Following reverse transcription, cDNA was quantified by using TaqMan RTQPCR assay (Custom TaqMan™ Small RNA Assay miRNA1 assay ID CSYPDRM and Custom TaqMan™ Small RNA Assay miRNA2 assay ID CT2W72H, Thermo Fisher scientific) and qualified standard lines. The QPCR was performed on 7500 Fast Real-Time PCR System (Thermo Fisher Scientific) and analysed on 7500 Software (v2.3) (Thermo Fisher Scientific).

### RNA Isolation and Quantification of Transcript Expression

Total RNA extraction was performed using AllPrep DNA/RNA Micro Kit (Qiagen) and following manufacturer's instructions. In brief, in order to detect specific transcripts expression levels, isolated RNA was reverse transcribed into cDNA by using a Maxima First Strand cDNA synthesis kit with DNase treatment (K1672, Thermofisher Scientific). Following reverse transcription, cDNA was quantified by using gene specific TaqMan RTQPCR assays (SEQ ID NO. 119 and 120 and 121) and housekeeping gene specific assays (ATP5B ID:Hs00969569_m1 (VIC), GAPDH ID:Hs02758991_g1 (FAM), ACTB ID:Hs01060665_g1 (FAM), HMBS Hs00609300_g1 (FAM), PSMB4 Hs00160598_g1 (FAM), Applied Biosystem). The QPCR was performed on QuantStudio™ 5 Real-Time PCR (Applied Biosystems) and analysed on QuantStudio™ Design & Analysis Software (Applied Biosystems).

### Endogenous Transcripts Lowering Following Transfection of pDNA

HEK293T cells were plated into 24-well tissue culture-treated plates at a density of 1E+05 cells/well. Cells were transfected in triplicates with 1 pg/cell of each of the plasmids using Lipofectamine 2000 (Thermo fisher Scientific) and collected 48 h post-transfection for RNA isolation. Total RNA was isolated from transfected HEK293T using Direct-zol RNA Microprep Kits (ZymoResearch) with a DNAse treatment step. The eluted RNA was quantified with Nanodrop and used for cDNA synthesis with the Maxima First Strand cDNA Synthesis Kit (ThermoFisher); subsequent Syber Green qPCR assay (QuantStudio™ 5) was performed with validated primer sets (table 1). The experiment was performed twice.

TABLE 1
Validated primer sets for the
Syber Green qPCR assay.

| SEQ | | | |
|---|---|---|---|
| ID | | | |
| NO | Gene | Type of gene | Primer |
| 82 | Gene 1 | Target transcript | 5′-ATGT |
| | | | TGGAGGA |
| | | | GCAGTGG |
| | | | TG-3′ |
| 83 | Gene 1 | Target transcript | 3′-TGTC |
| | | | AGGATCC |
| | | | ACAGGCA |
| | | | TA-5′ |
| 84 | Gene 5 | Target transcript | 5′-ACAG |
| | | | ATGCACC |
| | | | GACCAAA |
| | | | AC-3′ |
| 85 | Gene 5 | Target transcript | 3′-CCTG |
| | | | AGCCATC |
| | | | ATTTGCT |
| | | | TC-5′ |
| 86 | HMBS | Housekeeping gene | 5′-GGCA |
| | | | ATGCGGC |
| | | | TGCAA- |
| | | | 3′ |
| 87 | HMBS | Housekeeping gene | 3′-GGGT |
| | | | ACCCACG |
| | | | CGAATCA |
| | | | C-5′ |
| 88 | PSMB4 | Housekeeping gene | 5′-CATT |
| | | | CCGTCCA |
| | | | CTCCCGA |
| | | | TT-3′ |
| 89 | PSMB4 | Housekeeping gene | 3′-CGAA |
| | | | CTTAACG |
| | | | CCGAGGA |
| | | | CT-5′ |
| 90 | C1ORF43 | Housekeeping gene | 3′-ACGC |
| | | | CTTTCAA |
| | | | GGGTGTA |
| | | | CG-5′ |
| 91 | C1ORF43 | Housekeeping gene | 3′-CAAA |
| | | | GACCCCT |
| | | | GTCCCAT |
| | | | AGC-5′ |

### Dual-Luciferase® Reporter Assay

To assess reporter gene lowering by Dual-Luciferase® reporter assay (Promega), HEK293T cells were plated into 24-well tissue culture-treated plates at a density of 2E+04 or 1E+05 cellscells/well. Screening plasmids DNA (pDNA) (0.01, 0.05, 0.5 or 1 pg/cell) were co-transfected using Lipofectamine 2000 (Thermo Fisher Scientific), along with either miRNA1, miRNA5, miRNA6 or miRNA7 Dual-Luciferase® reporter vectors containing relevant miRNA binding sites (SEQ ID NO. 52, SEQ ID NO. 53, SEQ ID NO. 122 and SEQ ID NO. 123) in the 3′ UTR of the Renilla luciferase (RL) gene and a HSV-TK driven Firefly Luciferase (FL). The co-transfection was performed at 1:10 or 1:2 or 1:1 or 5:1 or 6:1 pDNA:Reporter ratios. The RL and FL activity was measured by Dual-Luciferase® reporter assay 48 h post-transfection. All transfections were performed as technical triplicates. The assay readouts were acquired in a GloMax Luminescence reader. Reporters lowering was measured as a decrease in the RL/FL activity ratio. The experiments were performed two or three times. Small RNA sequencing using next-generation sequencing (NGS) and data analysis

Total RNA was isolated from transfected HEK293T cells as described above. Small RNA sequencing was performed by GenomeScan BV (Leiden, Netherlands) using the NebNext small RNA library prep method (in vitro samples) or the Nextflex small RNA library preparation method (in vivo samples), including BluePippin size selection of the final library combined with Illumina NovaSeq6000 PE150 sequencing. The data were delivered and analyzed in-house using CLC Genomics Workbench Suit v20 (Qiagen). The trimmed small RNA reads were aligned against the human or mouse database of micro-RNAs (miRbase v22) and against a custom database containing the synthetic (pre-)miRNA sequences (SEQ ID NO. 28-41, 54, 104 and 105). The quantification of the miRNA1 to 6 and miR-144 derived sequences (guide and passenger strands where relevant) is expressed as a % of the number of counts/sequence versus the total number of small RNA counts.

### Results

### Design of Novel Scaffolds

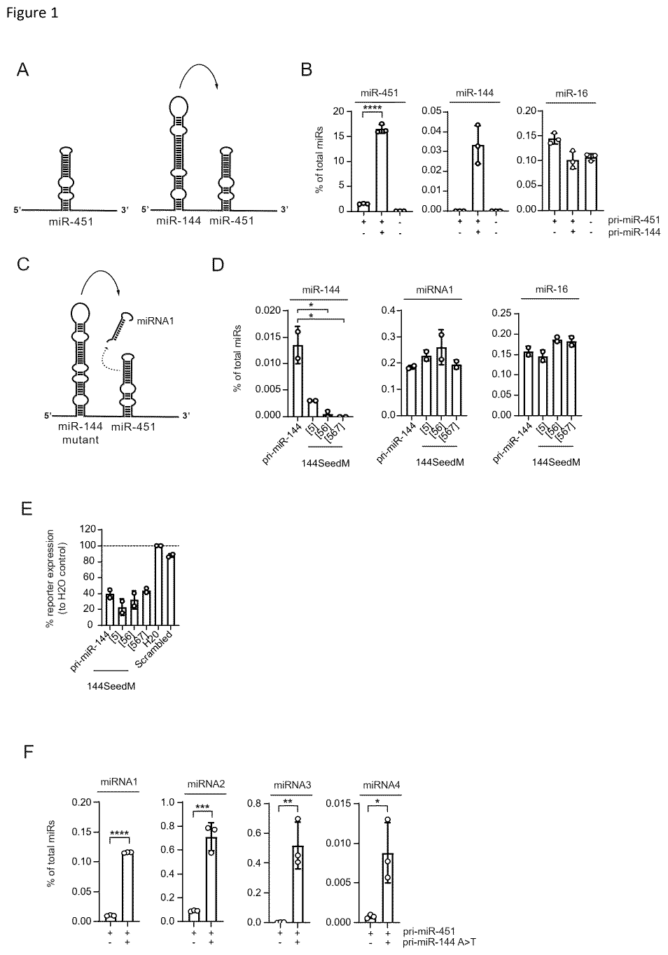

A series of scaffold alterations were made to the miR-451 and miR-144-miR-451 scaffolds in order to adapt the platform to a bi-or multi-miRNA expression system, allowing for improved single gene targeting and enabling multi-gene targeting. As presented in FIG. 1A, the system relies on either a single hairpin (miR-451) or a double hairpin (miR-144-miR-451) structure. The single hairpin structure is composed solely of the miR-451 microRNA scaffold for miRNA expression, whereas the double hairpin structure takes advantage of the miR-144-miR-451 microRNA cluster (Kretov et al. 2020, Molecular Cell 78, 317-328).). As described above, the later makes use of the helper function (represented by the black arrow in FIG. 1) of the microRNA miR-144 to enhance the processing and therefore the expression of miR-451 (Fang et al. 2020 Mol. Cell 78, 289-302). Indeed, small RNA sequencing revealed an augmented expression of a synthetic microRNA (SEQ ID NO. 6) from miR-451 when the later hairpin was in a context of a cluster involving miR-144 (FIG. 1B).

Since an exogenous expression of miR-144 would lead to side-effects e.g. miR-144 targets engagement (Huang et al. 2021, Journal of Cellular and Molecular Medicine 25(5): 2377-89; H. Li et al. 2016, Scientific Reports 6:1-12, Lin et al. 2020, Frontiers in Genetics 11:1-12), a novel strategy to introduce modifications within the scaffold in order to abrogate miR-144 expression was designed (FIG. 1C). Mutant versions of miR-144 hairpin were engineered, wherein base pairs at seed positions 5; 5 and 6; 5,6 and 7 from DROSHA cleavage site were mismatched using nucleotide substitutions, creating a bulge in the vicinity of the DROSHA cleavage site (generating 144 SeedM[5] variant miR-144 A>T (SEQ ID NO. 54), 144 SeedM[56] (SEQ ID NO. 126) and 144 SeedM[567] (SEQ ID NO. 127) mutants respectively). Each mutant miR-144 was clustered with miR-451 expressing a miRNA1 synthetic sequence (FIG. 1C). As expected, the introduced bulges disrupted miR-144 expression (FIG. 1D) (Shaohua Li et al., 2021 RNA Biology, 18:11, 1716-1726; Shaohua Li et al. 2020 Nature Communications 11(1) at various degrees however, with increasing disruptive efficiency with bulge complexity to almost undetectable levels with the triple mismatch (144 SeedM[567] mutant) (FIG. 1D, left graph). Interestingly, disruption of pri-miR-144 DROSHA cleavage improved miRNA1 expression from miR-451 (FIG. 1D). Indeed, miRNA1 expression increased in the SeedM[5] variant miR-144 A>T—as well as miRNA1 target reporter lowering (FIG. 1E), suggesting a mechanism whereby more DROSHA is liberated to operate onto miR-451. Notably, disrupting the miR-144 hairpin to a greater extent with a double or triple mismatch (144 SeedM[56] and 144 SeedM[567] mutants respectively), did not improve miRNA1 expression nor reporter lowering (FIG. 1D and FIG. 1E).

The expression values of an array of various synthetic miRNA sequences (miRNA1, miRNA2, miRNA3 and miRNA4) expressed from miR-451 scaffold (without and with miR-144A>T helper, scaffold A) (SEQ ID NO 58-65) were also analyzed, confirming an unexpected and tremendous improvement of miR-451 expressed synthetic miRNAs in the presence of miR-144A>T helper (FIG. 1F). Therefore, the use of mutant helper miR-144 enables to take advantage of the miR-144-miR-451 helper activity without expression of unwanted microRNAs, and allows for higher synthetic miRNA expression levels. miR-144A>T mutant-miR-451 cluster was therefore selected for use in the scaffold A technology, and subsequently exploited to develop next generation scaffolds (FIG. 2).

### Scaffolds B to E

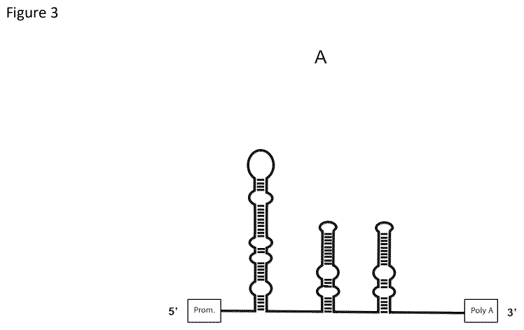

Next generation scaffolds were designed with the aim of expressing multiple synthetic miRNA sequences from one multi-hairpin structure. To this end, various multiplexed versions of Scaffold A (FIG. 2A) were created and tested for synthetic miRNA expression (FIG. 2B to 2E). In order to take advantage of an optimal miR-144-451 cluster assistance activity while avoiding repetitive regions and therefore recombination events, the 15 bp miR-451 5′ and 3′ flanking regions were conserved, and a 30 bp spacer sequence (SEQ ID NO. 48) was introduced between both miR-451 hairpins while conserving the human endogenous 92 bp region separating miR-144 from miR-451 (SEQ ID NO. 46) (FIG. 3) (Choi et al. 2015, Molecular Therapy 23(2): 310-20). The relative position of mutant miR-144 A>T and its helper effect on miR-451 expression was also investigated in the scaffolds B (SEQ ID NO 67-68) (FIG. 2B) and E (SEQ ID NO. 69-70) (FIG. 2E) where mutant miR-144A>T was placed at its original position 92 bp upfront 451 (FIG. 2B) or at equal distance (92 bp) between two miR-451 hairpins (FIG. 2E). An additional scaffold C (SEQ ID NO 71-72) (FIG. 2C) was designed to eventually rescue a loss of expression of the second more distal miR-451 hairpin; in said scaffold, a second mutant miR-144 was integrated in front of the second miR-451 hairpin. Finally, to assess the maximum helper effect of miR-144, additional miR-451 hairpins where integrated in scaffold D (SEQ ID NO. 73-74) (FIG. 2D) where up to four miR-451 hairpins were linked together.

### Functionality Tests of Scaffolds B to E

In order to test whether the above described scaffolds affect the expression of the integrated miRNA sequences, the extent of synthetic miRNA expression across the different scaffold designs was assessed. HEK293T cells were transfected, total RNA was isolated and small RNA sequencing on total RNA was performed.

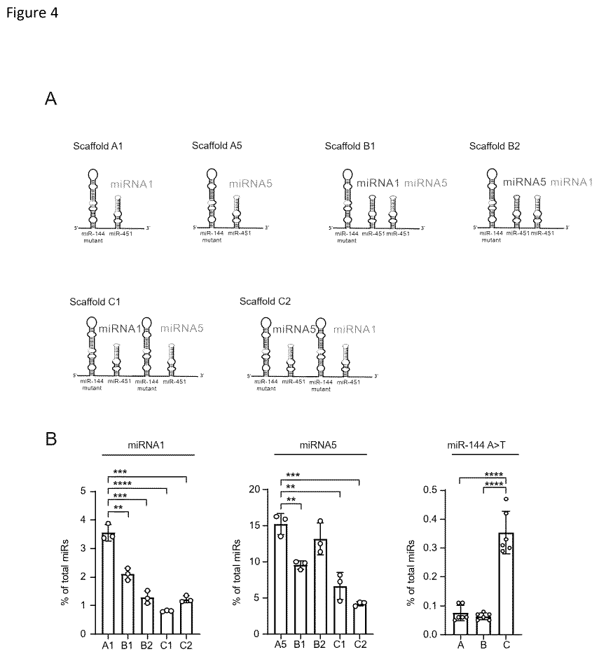

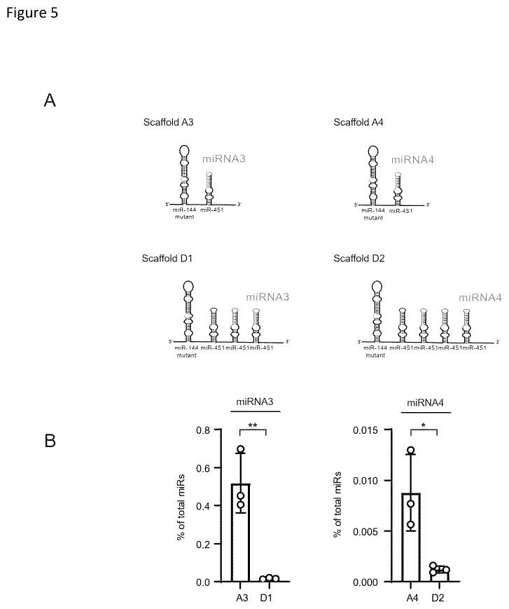

In order to evaluate the effect of the relative position of each integrated miRNA with respect to the miR-144A>T mutant on their expression levels, scaffold B was designed as two derivatives wherein miRNA1 and miRNA5 were each placed at a distal or proximal position to miR-144A>T helper (B1 and B2, respectively; FIG. 4A). Rescue scaffolds to B1 and B2 were also designed (scaffolds C1 and C2, respectively) to evaluate a potential rescue effect given by an additional helper hairpin (FIG. 4A). miRNA1 and miRNA5 expression was compared to their expression values from miR-144A>T-miR-451 scaffolds A1 or A5, respectively. The quantification data (as percentage of total miRs) showed a loss of expression for both miRNA1 and miRNA5 for scaffolds B and C, which was even more pronounced when the miRNA was integrated within the distal miR-451 hairpin (FIG. 4B). This loss of expression could not be rescued by addition of another helper miR-144A>T hairpin, which, surprisingly, seemed to aggravate the loss of expression of the first miRNA. Interestingly, quantification of miR-144A>T across scaffolds A, B and C showed a synergistic effect on expression values in scaffold C, suggesting that the helper effects might have been redirected onto the miR-144A>T rather than onto the miR-451 hairpins (FIG. 4B). Moreover, the addition of a third (scaffold D1) or fourth (scaffold D2) miR-451 hairpin (FIG. 5A) resulted in a impaired expression of the integrated miRNA compared to scaffold A-3 or A-4 (FIG. 5B).

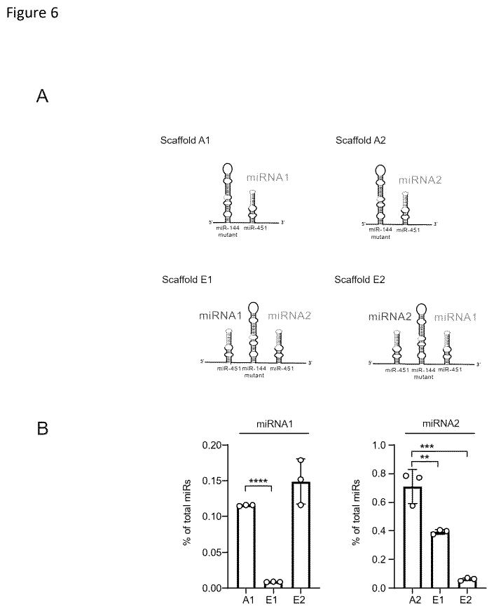

To assess whether a certain localization of helper miR-144A>T would benefit the expression of both miR-451 hairpins, miR-144A>T was re-localized from its original position 92 bp upfront two miR-451 hairpins to a middle position at equal distance of 92 bp between two miR-451 hairpins (scaffold E, FIG. 2E). Two derivatives of scaffold E were generated, placing either miRNA1 or miRNA2 before miR-144A>T helper (scaffold E1 and E2, respectively; FIG. 6A), and their expression as % of total miRs was evaluated by small RNA sequencing. Small RNA sequencing results showed a loss of expression of the miRNA located before miR-144A>T helper regardless of the miRNA sequence while the distal miRNA levels remained mostly unchanged (FIG. 6B).

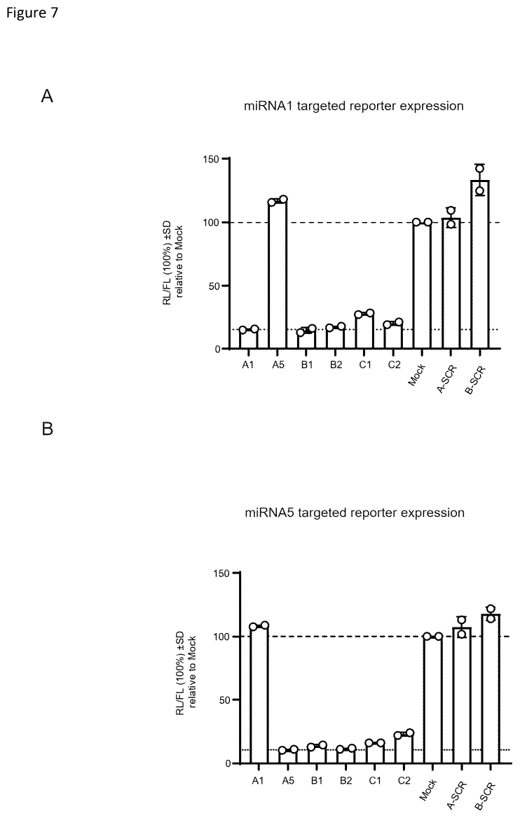

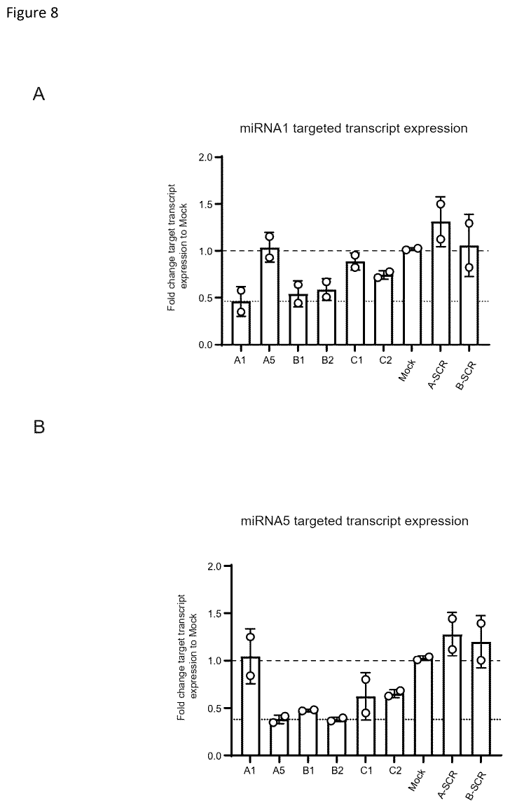

Next, individual scaffolds were tested for the functionality of the expressed synthetic miRNAs to lower its target sequence and compared to the A scaffolds. For this, screening plasmids expressing the different scaffolds were co-transfected along with a Dual-Luciferase® reporter vector containing miRNA1 or miRNA5 binding sites in the 3′ UTR of the Renilla luciferase gene, which activity was measured by Dual-Luciferase® reporter assay 48 h post-transfection. Both synthetic miRNA sequences within scaffolds B (Scaffolds B1 and B2) showed similar responses in luciferase reporter lowering to those observed when individually expressed within scaffolds A (Scaffolds A1 and A5), regardless of the more proximal or distal localization from the miR-144A>T helper hairpin (FIGS. 7A and 7B). Nonetheless, a slight loss of reporter lowering could be identified within scaffolds C (Scaffolds C1 and C2) for both expressed miRNA sequences, with a stronger trend for the sequence located between two miR-144 hairpins, in line with miRNA expression data (FIG. 7A Scaffold C1, FIG. 7B Scaffold C2 and FIG. 4). Dual-Luciferase® reporter assay results were consolidated with expression data for miRNA1 and miRNA5 targeting transcripts (FIGS. 8A and 8B, respectively).

Altogether, the data consolidates the use of Scaffold B as a mean to express two synthetic miRNAs and achieve lowering of two separate targets efficiently.

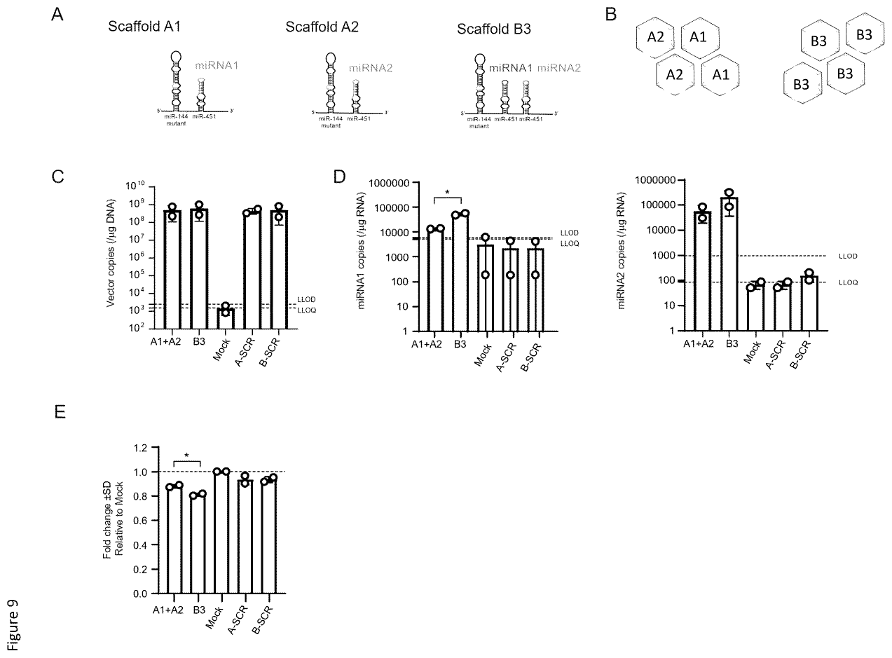

To validate the use of the scaffold B as a mean for augmented single target lowering where both expressed miRNAs share the same target, we designed scaffold B3 by combining the expression of miRNA1 with miRNA2 (FIG. 9A). To more closely mimic a gene therapy context, we cloned Scaffolds A1, A2 and B3 within an ITR plasmid and produced AAV9-A1, AAV9-A2 and AAV9-B3 viruses. Subsequently, cells were transduced with an identical MOI of an equi-mixture of AAV9-A1 and AAV9-A2, or AAV9-B3 alone (FIG. 9B). Comparable transduction efficiency in all conditions was assessed by vector copies quantification in both test conditions and controls (FIG. 9C) as well as expression of miRNA1 and miRNA2 (FIG. 9D). Interestingly, target transcript expression showed an augmented lowering effect with scaffold B3 compared to an equivalent MOI of two different AAVs (FIG. 9E), suggesting an additive value for the use of scaffold B3 in a context of single target lowering as well.

### Version 2 Scaffolds

In order to avoid the structural complexity of the above described scaffolds and to favour optimal miRNA expression, it was sought to take advantage not only of the miR-451 helper capacity of miR-144 but also of its potential as an expressing scaffold (FIG. 10A). As opposed to miR-451 (a non-canonical, DICER independent microRNA), miR-144 is a canonical microRNA and is processed through a DICER dependent pathway (Cheloufi et al. 2010, Nature 465(7298): 584-89; Cifuentes et al. 2010, Science 328(5986): 1694-98). Utilizing both miR-144 and miR-451 simultaneously as expressing scaffolds has the advantage of distributing the maturation of two synthetic miRNAs over two distinct microRNA pathways, therefore optimizing the expression of both mature miRNAs (Ma et al. 2014, Molecular Therapy—Nucleic Acids 3: e176).

Consequently, synthetic miRNA sequences within miR-144 were integrated in scaffolds F (SEQ ID NO. 77-81; 106). As a canonical microRNA, miR-144 processing generates a siRNA duplex following DROSHA and DICER activity; depending on the relative thermostabilities of the duplex ends, one or the other strand will be favoured for RISC loading and mRNA transcript targeting, whilst the other strand will be degraded (Medley et al. 2021, Wiley Interdisciplinary Reviews: RNA 12(3): 1-22). In the case of native miR-144, deep sequencing data show the 3p arm is the guide strand whereas the 5p arm is degraded (https://www.mirbase.org/cgi-bin/mirna_entry.pl?acc=MI0000460).

Because the passenger strand selection is sequence dependent, both 5p and 3p regions of miR-144 were designed as synthetic miRNA sequence recipients for miRNA5 (Scaffolds F1, FIG. 10B). Nucleotide modifications were introduced to adapt miRNA5 for miR-144 3p optimal guide expression (F1 3p19C design, with miRNA5 nucleotide19 A>C mutation) or, on the contrary, to adapt miRNA5 for miR-144 3p optimal passenger expression (F1 3p1G design, with miRNA5 nucleotide1 U>G mutation). The resulting miR-144 expressing scaffolds are shown in FIG. 10C (and SEQ ID NO. 37-41).

The expression capacity of miR-144 was validated and the stoichiometries between guide and passenger strands were determined and represented as the Guide/Passenger ratio (G/P ratio) of miRNA5 over miRNA5*, as shown in FIG. 10D left graph. Scaffold F1 5p had the most favourable G/P ratio (12.4). The nucleotide modification in the F1 3p19C design indeed increased guide strand expression over passenger strand as shown by an increased G/P ratio of 17-fold compared to the non-modified F1 3p design (5.1 vs 0.3, respectively). As opposed, the F1 3p1G design had a 7.5-fold lower G/P ratio compared to F1 3p (0.04 versus 0.3 respectively), suggesting a predominance of passenger strand expression as anticipated. Importantly, the guide strand selection modifications in the miR-144 hairpin did not affect miRNA1 expression from miR-451 (FIG. 10D, right graph).

Following miRNA quantification by small RNA sequencing, we looked into the isomiR population for each of the synthetic miRNAs expressed (FIG. 10E). Notably, miRNA1 processing from miR-451 was more heterogenous compared to miR-144 expressed miRNA. The main expressed form was a 25 nt trimmed sequence which represented over 60% of all miRNA1 isomiRs with a distribution ranging from 17 nt to 30 nt trimmed miRNA1. However and importantly, the isomiR distribution among all four scaffolds was similar suggesting that the nature of miR-144 does not affect the trimming of miR-451. On the contrary, processing of miRNA5 varied greatly among the scaffolds. While F1 5p produced up to 90% of 23 nt miRNA5, F1 3p produced overall 1, 2 or 3 nt shorter forms of miRNA5 (22.5%, 55% and 15.6% respectively). Interestingly, modifying the G/P ratios also modified the isomiR distribution of miRNA5 with an increase in the 20 nt isomiR in F1 3p19C and an increase in the 22 nt isomiR in F1 3p 1G.

Finally, to validate the functionality of the expressed miRNAs, Dual-Luciferase® reporter assays were performed as described previously. In brief, plasmids were co-transfected with a Dual-Luciferase® reporter vector containing miRNA1 or miRNA5 binding sites in the Renilla luciferase gene with a read-out measurement 48 h post-transfection. All Scaffolds F1 showed an optimal lowering of the miRNA1 targeting reporter suggesting an optimal miRNA1 expression from the miR-451 hairpin (FIG. 10 F right graph), while the lowering of the miRNA5 targeting reporter correlated with the G/P ratio (FIGS. 10D and 10F left graph). Dual-Luciferase® reporter assay results were consolidated with expression data for miRNA1 and miRNA5 targeted transcripts (FIG. 10G left and right graphs, respectively).

In order to further validate the use of Scaffold F as a therapeutic mean, we designed Scaffold F2 with a set of new synthetic miRNAs; miRNA6 inserted in miR-144 5p and miRNA7 inserted within miR-451 (FIG. 11A). In vitro transfection validated the functionality of Scaffold F2, with expression of both miRNA6 and miRNA7 and a G/P ratio of 1.7 (FIG. 11B). Analysis of the isomiR profiles for both synthetic miRNAs showed a dominance for 23 nt and 28 nt isomiRs for miRNA6 and miRNA7 respectively (FIG. 10C). Dual-Luciferase® reporter assay validated the dual lowering capacity of the scaffold F2 (FIG. 11D). Following in vitro validation of the scaffold, we proceeded to in vivo validation. Scaffold F2 was packaged within a AAV5 capsid and delivered in mice brains by direct intra-striatal injection (FIG. 11E). Striatal tissue was harvested 12 weeks post-injection to evaluate the transduction efficacy as well as the synthetic miRNA expression profiles. Interestingly, for an average of 1.5e+7 vector AAV5 copies/μg striatal DNA (FIG. 11F), we were able to quantify a miRNA6 and miRNA7 expression of total microRNAs up to 3% and 0.6% respectively, where expression from miR-144 5p performed better than expression from miR-451 (FIG. 11G left graph). Surprisingly, the miRNA6 guide/passenger ratio in this context showed a remarkable improvement 15 times over in vitro context (26 versus 1.7 respectively), suggesting a discrepancy in the strands stability over time in vivo (FIG. 11G right graph). Importantly, the in vivo miRNA processing analysis recapitulated the same isomiR profiles of both synthetic miRNAs (FIG. 11H) with more than 95% of exact 5′ seed sequence expression (FIG. 11I) suggesting an extreme DROSHA fidelity to generate active microRNA species.

Altogether, miRNA expression data and functionality assays support that 1) two distinct synthetic miRNAs can be expressed from one same scaffold, 2) modifying miR-144 sequence does not affect its helper function on miR-451, 3) both 3p and 5p synthetic miRNA expression from miR-144 can lead to optimal target lowering and if not, 4) modulation of the guide/passenger ratio can be achieved to enrich for the desired strand and 5) desired expression levels of both synthetic miRNA expressed from miR-144 and miR-451 can be achieved in vivo following local rAAV injection.

1. A nucleic acid comprising a sequence encoding a first RNA and a sequence encoding a second RNA, wherein:
i. the first RNA comprises a first guide sequence substantially complementary to a first target sequence in a first transcript encoded by a first gene;
ii. the second RNA comprises a second guide sequence substantially complementary to a second target sequence in a second transcript encoded by a second gene;
iii. the first RNA and the second RNA each comprise a hairpin; and
iv. the first RNA and the second RNA each comprise a sequence selected from the group consisting of: SEQ ID NO. 93; SEQ ID NO. 94; and variants thereof.

2. The nucleic acid according to claim 1, wherein, in a 5′ to 3′ direction, the sequence encoding the first RNA is followed by a first spacer comprising at least 15 nucleotides, which spacer is followed by the sequence encoding the second RNA.

3. The nucleic acid according to claim 1, wherein the first and the second RNA comprise SEQ ID NO. 93 or a variant thereof.

4. The nucleic acid according to claim 3, wherein the nucleic acid further comprises a sequence encoding a third RNA, and wherein the third RNA comprises SEQ ID NO. 54 or a variant thereof.

5. The nucleic acid according to claim 4, wherein, in a 5′ to 3′ direction, the sequence encoding the third RNA is followed by a second spacer comprising at least 15 nucleotides, which second spacer is followed by the sequence encoding the first RNA, followed by the first spacer and the sequence encoding the second RNA.

6. The nucleic acid according to claim 3, wherein the third RNA is mutated to reduce processing and/or expression of the third RNA.

7. The nucleic acid according to claim 1, wherein the first RNA comprises: SEQ ID NO. 94 or a variant thereof; and the second RNA comprises SEQ ID NO. 93 or a variant thereof.

8. The nucleic acid according to claim 1, wherein at least one of the target sequences is comprised in an antisense RNA transcript encoded by a human gene.

9. The nucleic acid according to claim 1, wherein the first and the second gene are the same gene.

10. The nucleic acid according to claim 9, wherein the first and the second target sequences are the same.

11. The nucleic acid according to claim 9, wherein the first and the second target sequences are different target sequences.

12. The nucleic acid according to claim 1, wherein the first and the second gene are different genes.

13. An expression cassette comprising a nucleic acid, wherein the expression cassette is a DNA molecule, and
wherein the nucleic acid comprises a sequence encoding a first RNA and a sequence encoding a second RNA, wherein:
i. the first RNA comprises a first guide sequence substantially complementary to a first target sequence in a first transcript encoded by a first gene;
ii. the second RNA comprises a second guide sequence substantially complementary to a second target sequence in a second transcript encoded by a second gene;
iii. the first RNA and the second RNA each comprise a hairpin; and
iv. the first RNA and the second RNA each comprise a sequence selected from the group consisting of: SEQ ID NO. 93; SEQ ID NO. 94; and variants thereof.

14. The expression cassette according to claim 13, wherein the nucleic acid is operably linked to a promoter and optionally to a poly-A signal.

15. The expression cassette according to claim 13, wherein the expression cassette is flanked by at least one AAV Inverted Terminal Repeat (ITR).

16. An adeno-associated virus (AAV) vector comprising an expression cassette, wherein the expression cassette is a DNA molecule,
wherein the expression cassette comprises a nucleic acid, and
wherein the nucleic acid comprises a sequence encoding a first RNA and a sequence encoding a second RNA, wherein:
i. the first RNA comprises a first guide sequence substantially complementary to a first target sequence in a first transcript encoded by a first gene;
ii. the second RNA comprises a second guide sequence substantially complementary to a second target sequence in a second transcript encoded by a second gene;
iii. the first RNA and the second RNA each comprise a hairpin; and
iv. the first RNA and the second RNA each comprise a sequence selected from the group consisting of: SEQ ID NO. 93; SEQ ID NO. 94; and variants thereof.

17. The AAV vector according to claim 16, comprising an AAV5 or AAV9 capsid protein.

18.-22. (canceled)


SEQUENCE LISTING (127 sequences)

SEQ ID NO: 1 - DNA, 22 bp, synthetic construct
  misc_feature 1..22: note = miRNA1
  source 1..22: mol_type = other DNA
tgctctttggtcttctcagcca

SEQ ID NO: 2 - DNA, 22 bp, synthetic construct
  misc_feature 1..22: note = miRNA2
  source 1..22: mol_type = other DNA
ttgcccaactggtcctttttga

SEQ ID NO: 3 - DNA, 22 bp, synthetic construct
  misc_feature 1..22: note = miRNA3
  source 1..22: mol_type = other DNA
tccttggcctttgaaagtcctt

SEQ ID NO: 4 - DNA, 22 bp, synthetic construct
  misc_feature 1..22: note = miRNA4
  source 1..22: mol_type = other DNA
ttggttttctcagcagcagcca

SEQ ID NO: 5 - DNA, 22 bp, synthetic construct
  misc_feature 1..22: note = miRNA5
  source 1..22: mol_type = other DNA
tctggaactaccttgcatactt

SEQ ID NO: 6 - DNA, 21 bp, synthetic construct
  misc_feature 1..21: note = miRNA6
  source 1..21: mol_type = other DNA
aaggacttgagggactcgaag

SEQ ID NO: 7 - DNA, 22 bp, synthetic construct
  misc_feature 1..22: note = miSCR1
  source 1..22: mol_type = other DNA
gtagttctattagcgcttacta

SEQ ID NO: 8 - DNA, 22 bp, synthetic construct
  misc_feature 1..22: note = miSCR2
  source 1..22: mol_type = other DNA
gcgacttgtaacgtcagaggcg

SEQ ID NO: 9 - DNA, 22 bp, synthetic construct
  misc_feature 1..22: note = miRNA5-5p
  source 1..22: mol_type = other DNA
tctggaactaccttgcatactt

SEQ ID NO: 10 - DNA, 20 bp, synthetic construct
  misc_feature 1..20: note = miRNA5-3P
  source 1..20: mol_type = other DNA
tctggaactaccttgcatac

SEQ ID NO: 11 - DNA, 20 bp, synthetic construct
  misc_feature 1..20: note = miRNA5-3P-19C
  source 1..20: mol_type = other DNA
tctggaactaccttgcatcc

SEQ ID NO: 12 - DNA, 20 bp, synthetic construct
  misc_feature 1..20: note = miRNA5-3P-1G
  source 1..20: mol_type = other DNA
gctggaactaccttgcatac

SEQ ID NO: 13 - DNA, 20 bp, synthetic construct
  misc_feature 1..20: note = miSCR2-3p
  source 1..20: mol_type = other DNA
gcgacttgtaacgtcagagg

SEQ ID NO: 14 - DNA, 18 bp, synthetic construct
  misc_feature 1..18: note = miRNA1 complementary
  source 1..18: mol_type = other DNA
tgagaagaccaaagagcg

SEQ ID NO: 15 - DNA, 18 bp, synthetic construct
  misc_feature 1..18: note = miRNA2 complementary
  source 1..18: mol_type = other DNA
aaaggaccagttgggcac

SEQ ID NO: 16 - DNA, 18 bp, synthetic construct
  misc_feature 1..18: note = miRNA3 complementary
  source 1..18: mol_type = other DNA
actttcaaaggccaaggc

SEQ ID NO: 17 - DNA, 18 bp, synthetic construct
  misc_feature 1..18: note = miRNA4 complementary
  source 1..18: mol_type = other DNA
tgctgctgagaaaaccac

SEQ ID NO: 18 - DNA, 16 bp, synthetic construct
  misc_feature 1..16: note = miRNA5 complementary
  source 1..16: mol_type = other DNA
atgcaaggtttccagc

SEQ ID NO: 19 - DNA, 18 bp, synthetic construct
  misc_feature 1..18: note = miRNA6 complementary
  source 1..18: mol_type = other DNA
gagtccctcaagtcctct

SEQ ID NO: 20 - DNA, 18 bp, synthetic construct
  misc_feature 1..18: note = miSCR1 complementary
  source 1..18: mol_type = other DNA
aagcgctaatagaactag

SEQ ID NO: 21 - DNA, 18 bp, synthetic construct
  misc_feature 1..18: note = miSCR2 complementary
  source 1..18: mol_type = other DNA
tctgacgttacaagtcga

SEQ ID NO: 22 - DNA, 20 bp, synthetic construct
  misc_feature 1..20: note = miRNA5 5p complementary
  source 1..20: mol_type = other DNA
gtatgcaagtagttccaaac

SEQ ID NO: 23 - DNA, 21 bp, synthetic construct
  misc_feature 1..21: note = miRNA5 3p complementary
  source 1..21: mol_type = other DNA
gtctgcaaggtcagttccaga

SEQ ID NO: 24 - DNA, 22 bp, synthetic construct
  misc_feature 1..22: note = miRNA5-19C 3p complementary
  source 1..22: mol_type = other DNA
gctgcaaggtcagttccagatg

SEQ ID NO: 25 - DNA, 22 bp, synthetic construct
  misc_feature 1..22: note = miRNA5-1G 3p complementary
  source 1..22: mol_type = other DNA
tctgcaaggtcagttccagctg

SEQ ID NO: 26 - DNA, 22 bp, synthetic construct
  misc_feature 1..22: note = miSCR2 3p complementary
  source 1..22: mol_type = other DNA
cgctgacgttcacaagtcgccg

SEQ ID NO: 27 - DNA, 72 bp, synthetic construct
  misc_feature 1..72: note = human pri-mir-451 sequence
  source 1..72: mol_type = other DNA
cttgggaatggcaaggaaaccgttaccattactgagttcagtaatggtaacggttctcttgctatacccaga

SEQ ID NO: 28 - DNA, 72 bp, synthetic construct
  misc_feature 1..72: note = miRNA1 in mir-451 scaffold
  source 1..72: mol_type = other DNA
cttgggaatggcaaggtgctctttggtcttctcagccatgagaagaccaaagagcgtcttgctatacccaga

SEQ ID NO: 29 - DNA, 72 bp, synthetic construct
  misc_feature 1..72: note = miRNA2 in mir-451 scaffold
  source 1..72: mol_type = other DNA
cttgggaatggcaaggttgcccaactggtcctttttgaaaaggaccagttgggcactcttgctatacccaga

SEQ ID NO: 30 - DNA, 72 bp, synthetic construct
  misc_feature 1..72: note = miRNA3 in mir-451 scaffold
  source 1..72: mol_type = other DNA
cttgggaatggcaaggtccttggcctttgaaagtccttactttcaaaggccaaggctcttgctatacccaga

SEQ ID NO: 31 - DNA, 72 bp, synthetic construct
  misc_feature 1..72: note = miRNA4 in mir-451 scaffold
  source 1..72: mol_type = other DNA
cttgggaatggcaaggttggttttctcagcagcagccatgctgctgagaaaaccactcttgctatacccaga

SEQ ID NO: 32 - DNA, 72 bp, synthetic construct
  misc_feature 1..72: note = miRNA5 in mir-451 scaffold
  source 1..72: mol_type = other DNA
cttgggaatggcaaggtctggaactaccttgcatacttatgcaaggtagttccagctcttgctatacccaga

SEQ ID NO: 33 - DNA, 72 bp, synthetic construct
  misc_feature 1..72: note = miRNA6 in mir-451 scaffold
  source 1..72: mol_type = other DNA
cttgggaatggcaaggaaggacttgagggactcgaagacgagtccctcaagtcctctcttgctatacccaga

SEQ ID NO: 34 - DNA, 72 bp, synthetic construct
  misc_feature 1..72: note = miSCR1 in mir-451 scaffold
  source 1..72: mol_type = other DNA
cttgggaatggcaagggtagttctattagcgcttactaaagcgctaatagaactaatcttgctatacccaga

SEQ ID NO: 35 - DNA, 72 bp, synthetic construct
  misc_feature 1..72: note = miSCR2 in mir-451 scaffold
  source 1..72: mol_type = other DNA
cttgggaatggcaagggcgacttgtaacgtcagaggcgtctgacgttacaagtcgatcttgctatacccaga

SEQ ID NO: 36 - DNA, 86 bp, synthetic construct
  misc_feature 1..86: note = human pri-mir-144 sequence
  source 1..86: mol_type = other DNA
tggggccctggctgggatatcatcatatactgtaagtttgcgatgagacactacagtatagatgatgtactagtccgggcaccccc

SEQ ID NO: 37 - DNA, 86 bp, synthetic construct
  misc_feature 1..86: note = miRNA5 in mir-144-5p scaffold
  source 1..86: mol_type = other DNA
tggggccctggctgtctggaactaccttgcatactttctgcgatgagaaccgtatgcaagtagttccaaacagtccgggcaccccc

SEQ ID NO: 38 - DNA, 86 bp, synthetic construct
  misc_feature 1..86: note = miRNA5 in mir-144-3p scaffold
  source 1..86: mol_type = other DNA
tggggccctggctgtctgcaaggtcagttccagatgtttgcgatgagaccctctggaactaccttgcatacagtccgggcaccccc

SEQ ID NO: 39 - DNA, 86 bp, synthetic construct
  misc_feature 1..86: note = miRNA5 A>C mutant in mir-144-3p scaffold
  source 1..86: mol_type = other DNA
tggggccctggctggctgcaaggtcagttccagatgtttgcgatgagaccctctggaactaccttgcatccagtccgggcaccccc

SEQ ID NO: 40 - DNA, 57 bp, synthetic construct
  misc_feature 1..57: note = miRNA5 T>G mutant in mir-144-3p scaffold
  source 1..57: mol_type = other DNA
tggggccctggctgtctgcaaggtcagttccagctgtttgcgatgagacccgctgga

SEQ ID NO: 41 - DNA, 86 bp, synthetic construct
  misc_feature 1..86: note = miSCR2 in mir-144-3p scaffold
  source 1..86: mol_type = other DNA
tggggccctggctccgctgacgttcacaagtcgccgtttgcgatgagacaagcgacttgtaacgtcagaggagtccgggcaccccc

SEQ ID NO: 42 - DNA, 157 bp, synthetic construct
  misc_feature 1..157: note = scaffold mir-451 5' flanking region
  source 1..157: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggca

SEQ ID NO: 43 - DNA, 28 bp, synthetic construct
  misc_feature 1..28: note = Scaffolds A, B, C, D and F 5' flanking region
  source 1..28: mol_type = other DNA
atcggcgctatgcttcctgtgcccccag

SEQ ID NO: 44 - DNA, 28 bp, synthetic construct
  misc_feature 1..28: note = Scaffolds E 5' flanking region
  source 1..28: mol_type = other DNA
atcggcgctatgcctgactgccagggca

SEQ ID NO: 45 - DNA, 205 bp, synthetic construct
  misc_feature 1..205: note = mir-451 and scaffolds A - F 3' flanking region
  source 1..205: mol_type = other DNA
aaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 46 - DNA, 92 bp, synthetic construct
  misc_feature 1..92: note = human endogenous 92 bp spacer region separating mir-144-mir-451 hairpins
  source 1..92: mol_type = other DNA
agctctggagcctgacaaggaggacaggagagatgctgcaagcccaagaagctctctgctcagcctgtcacaacctactgactgccagggca

SEQ ID NO: 47 - DNA, 92 bp, synthetic construct
  misc_feature 1..92: note = 92 bp spacer region separating mir-451-144 hairpins
  source 1..92: mol_type = other DNA
acgggaccgtcagtcatccaacactgtccgactcgtctctcgaagaacccgaacgtcgtagagaggacaggaggaacagtccgaggtctcga

SEQ ID NO: 48 - DNA, 30 bp, synthetic construct
  misc_feature 1..30: note = 30 bp spacer sequence between mir-451-mir-451 hairpins
  source 1..30: mol_type = other DNA
aaacgtgccaggaagctgactgccagggca

SEQ ID NO: 49 - DNA, 2438 bp, synthetic construct
  misc_feature 1..2438: note = pUC screening vector
  source 1..2438: mol_type = other DNA
aagctttagagctcatggcgcgcctaggccttgacggccttccgccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggccactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaggcaccccaggctttacccgaacgaccgagcgcagcgagtcagtgagcgaggaagcggagagcgcccaatacgcaaggaaacagctatgaccatgttaatgcagctggcacgacaggtttcccgactggaaagcgggcagtgagcggaaggcccatgaggccagttaattaacgagctcctagttattaatagtaatcaattacggggtcattagttcatagcccatatatggagttccg

SEQ ID NO: 50 - DNA, 1602 bp, synthetic construct
  misc_feature 1..1602: note = CAG promoter
  source 1..1602: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaagtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgcgccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 51 - DNA, 269 bp, synthetic construct
  misc_feature 1..269: note = Bovine growth hormone polyadenylation signal (bgh polyA)
  source 1..269: mol_type = other DNA
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 52 - DNA, 612 bp, synthetic construct
  misc_feature 1..612: note = Insert dual luciferase reporter with miRNA1 targeted sequence
  source 1..612: mol_type = other DNA
ggcgatcgctcgacatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtatgcggccgctggccgcaataaaatatctttattttcattacatctgtgtgttggttttttgtgtg

SEQ ID NO: 53 - DNA, 1086 bp, synthetic construct
  misc_feature 1..1086: note = Insert dual luciferase reporter with miRNA5 targeted sequence
  source 1..1086: mol_type = other DNA
atggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataa

SEQ ID NO: 54 - DNA, 86 bp, synthetic construct
  misc_feature 1..86: note = mir-144 A >T mutant helper
  source 1..86: mol_type = other DNA
tggggccctggctgggatttcatcatatactgtaagtttgcgatgagacactacagtatagatgatgtactagtccgggcaccccc

SEQ ID NO: 55 - DNA, 434 bp, synthetic construct
  misc_feature 1..434: note = miRNA6 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 56 - DNA, 483 bp, synthetic construct
  misc_feature 1..483: note = miRNA6 in mir-451 cassette with mir-144WT
  source 1..483: mol_type = other DNA
atcggcgctatgcttcctgtgcccccagtggggccctggctgggatatcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 57 - DNA, 483 bp, synthetic construct
  misc_feature 1..483: note = miRNA6 in mir-451 cassette with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 58 - DNA, 434 bp, synthetic construct
  misc_feature 1..434: note = miRNA1 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 59 - DNA, 434 bp, synthetic construct
  misc_feature 1..434: note = miRNA2 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 60 - DNA, 434 bp, synthetic construct
  misc_feature 1..434: note = miRNA3 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 61 - DNA, 434 bp, synthetic construct
  misc_feature 1..434: note = miRNA4 in mir-451 cassette
  source 1..434: mol_type = other DNA
atcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 62 - DNA, 483 bp, synthetic construct
  misc_feature 1..483: note = Cassette A1, miRNA1 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 63 - DNA, 483 bp, synthetic construct
  misc_feature 1..483: note = Cassette A2, miRNA2 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 64 - DNA, 483 bp, synthetic construct
  misc_feature 1..483: note = Cassette A3, miRNA3 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 65 - DNA, 483 bp, synthetic construct
  misc_feature 1..483: note = Cassette A4, miRNA4 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 66 - DNA, 483 bp, synthetic construct
  misc_feature 1..483: note = Cassette A5, miRNA5 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtctggaactaccttgcatacttatgcaaggtagttccagctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 67 - DNA, 585 bp, synthetic construct
  misc_feature 1..585: note = Cassette B1, miRNA1 and miRNA5 in cassette B
  source 1..585: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggtctggaactaccttgcatacttatgcaaggtagttccagctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 68 - DNA, 585 bp, synthetic construct
  misc_feature 1..585: note = Cassette B2, miRNA5 and miRNA1 in cassette B
  source 1..585: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtctggaactaccttgcatacttatgcaaggtagttccagctcttgctatacccagaaaacgtgccaggaagctgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 69 - DNA, 647 bp, synthetic construct
  misc_feature 1..647: note = Cassette E1, miRNA1 and miRNA2 in cassette E
  source 1..647: mol_type = other DNA
atcggcgctatgcctgactgccagggcacttgggaatggcaaggtgctctttggtcttctcagccatgagaagaccaaagagcgtcttgctatacccagaacgggaccgtcagtcatccaacactgtccgactcgtctctcgaagaacccgaacgtcgtagagaggacaggaggaacagtccgaggtctcgatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 70 - DNA, 647 bp, synthetic construct
  misc_feature 1..647: note = Cassette E2, miRNA2 and miRNA1 in cassette E
  source 1..647: mol_type = other DNA
atcggcgctatgcctgactgccagggcacttgggaatggcaaggttgcccaactggtcctttttgaaaaggaccagttgggcactcttgctatacccagaacgggaccgtcagtcatccaacactgtccgactcgtctctcgaagaacccgaacgtcgtagagaggacaggaggaacagtccgaggtctcgatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 71 - DNA, 825 bp, synthetic construct
  misc_feature 1..825: note = Cassette C1, miRNA1 and miRNA5 in cassette C
  source 1..825: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaacgggaccgtcagtcatccaacactgtccgactcgtctctcgaagaacccgaacgtcgtagagaggacaggaggaacagtccgaggtctcgatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtctggaactaccttgcatacttatgcaaggtagttccagctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 72 - DNA, 825 bp, synthetic construct
  misc_feature 1..825: note = Cassette C2, miRNA5 and miRNA1 in cassette C
  source 1..825: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtctggaactaccttgcatacttatgcaaggtagttccagctcttgctatacccagaacgggaccgtcagtcatccaacactgtccgactcgtctctcgaagaacccgaacgtcgtagagaggacaggaggaacagtccgaggtctcgatggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 73 - DNA, 687 bp, synthetic construct
  misc_feature 1..687: note = Cassette D1, miRNA3 in cassette D1
  source 1..687: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggttgcccaactggtcctttttgaaaaggaccagttgggcactcttgctatacccagaaaacgtgccaggaagctgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 74 - DNA, 789 bp, synthetic construct
  misc_feature 1..789: note = Cassette D2, miRNA4 in cassette D2
  source 1..789: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggttgcccaactggtcctttttgaaaaggaccagttgggcactcttgctatacccagaaaacgtgccaggaagctgactgccagggcacttgggaatggcaaggtccttggcctttgaaagtccttactttcaaaggccaaggctcttgctatacccagaaaacgtgccaggaagctgactgccagggcacttgggaatggcaaggttggttttctcagcagcagccatgctgctgagaaaaccactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 75 - DNA, 483 bp, synthetic construct
  misc_feature 1..483: note = Cassette A SCR, miSCR1 in mir-451 scaffold with mir-144A>T
  source 1..483: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 76 - DNA, 585 bp, synthetic construct
  misc_feature 1..585: note = Cassette B SCR, miSCR1 and miSCR2 in cassette B
  source 1..585: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagctgactgccagggcacttgggaatggcaagggcgacttgtaacgtcagaggcgtctgacgttacaagtcgatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 77 - DNA, 483 bp, synthetic construct
  misc_feature 1..483: note = Cassette F 5p, miRNA5 and miRNA1 in cassette F
  source 1..483: mol_type = other DNA
atcggcgctatgcttcctgtgcccccagtggggccctggctgtctggaactaccttgcatactttctgcgatgagaaccgtatgcaagtagttccaaacagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 78 - DNA, 483 bp, synthetic construct
  misc_feature 1..483: note = Cassette F 3p, miRNA5 and miRNA1 in cassette F
  source 1..483: mol_type = other DNA
atcggcgctatgcttcctgtgcccccagtggggccctggctgtctgcaaggtcagttccagatgtttgcgatgagaccctctggaactaccttgcatacagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 79 - DNA, 483 bp, synthetic construct
  misc_feature 1..483: note = Cassette F 3p19C, miRNA5 A>C mutant and miRNA1 in cassette F
  source 1..483: mol_type = other DNA
atcggcgctatgcttcctgtgcccccagtggggccctggctggctgcaaggtcagttccagatgtttgcgatgagaccctctggaactaccttgcatccagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 80 - DNA, 483 bp, synthetic construct
  misc_feature 1..483: note = Cassette F 3p1G, miRNA5 T>G mutant and miRNA1 in cassette F
  source 1..483: mol_type = other DNA
atcggcgctatgcttcctgtgcccccagtggggccctggctgtctgcaaggtcagttccagctgtttgcgatgagacccgctggaactaccttgcatacagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 81 - DNA, 483 bp, synthetic construct; identical to SEQ ID NO: 80
  misc_feature 1..483: note = Cassette F SCR, miSCR2 and miSCR1 in cassette F
  source 1..483: mol_type = other DNA

SEQ ID NO: 82 - DNA, 20 bp, synthetic construct
  misc_feature 1..20: note = SNCA2 primer 1
  source 1..20: mol_type = other DNA
atgttggaggagcagtggtg

SEQ ID NO: 83 - DNA, 20 bp, synthetic construct
  misc_feature 1..20: note = SNCA2 primer 2
  source 1..20: mol_type = other DNA
tgtcaggatccacaggcata

SEQ ID NO: 84 - DNA, 20 bp, synthetic construct
  misc_feature 1..20: note = ATXN3 primer 1
  source 1..20: mol_type = other DNA
acagatgcaccgaccaaaac

SEQ ID NO: 85 - DNA, 20 bp, synthetic construct
  misc_feature 1..20: note = ATXN3 primer 2
  source 1..20: mol_type = other DNA
cctgagccatcatttgcttc

SEQ ID NO: 86 - DNA, 16 bp, synthetic construct
  misc_feature 1..16: note = HMBS primer 1
  source 1..16: mol_type = other DNA
ggcaatgcggctgcaa

SEQ ID NO: 87 - DNA, 19 bp, synthetic construct
  misc_feature 1..19: note = HMBS primer 2
  source 1..19: mol_type = other DNA
gggtacccacgcgaatcac

SEQ ID NO: 88 - DNA, 20 bp, synthetic construct
  misc_feature 1..20: note = PSMB4 primer 1
  source 1..20: mol_type = other DNA
cattccgtccactcccgatt

SEQ ID NO: 89 - DNA, 20 bp, synthetic construct
  misc_feature 1..20: note = PSMB4 primer 2
  source 1..20: mol_type = other DNA
cgaacttaacgccgaggact

SEQ ID NO: 90 - DNA, 20 bp, synthetic construct
  misc_feature 1..20: note = C1ORF43 primer 1
  source 1..20: mol_type = other DNA
acgcctttcaagggtgtacg

SEQ ID NO: 91 - DNA, 21 bp, synthetic construct
  misc_feature 1..21: note = C1ORF43 primer 1
  source 1..21: mol_type = other DNA
caaagacccctgtcccatagc

SEQ ID NO: 92 - DNA, 16 bp, synthetic construct
  misc_feature 1..16: note = First stem sequence within mir-451 hairpin
  source 1..16: mol_type = other DNA
cttgggaatggcaagg

SEQ ID NO: 93 - DNA, 72 bp, synthetic construct
  misc_feature 1..72: note = miR-451-derived scaffold
  misc_feature 17..55: note = n is a, c, g, t or u
  source 1..72: mol_type = other DNA
cttgggaatggcaaggnnnnnnnnnnnnnnnnnnnnnnnnnnnnnnnnnnnnnnnctcttgctatacccaga

SEQ ID NO: 94 - DNA, 86 bp, synthetic construct
  misc_feature 1..86: note = miR-144-derived scaffold
  misc_feature 15..36: note = n is a, c, g, t or u
  misc_feature 52..71: note = n is a, c, g, t or u
  source 1..86: mol_type = other DNA
tggggccctggctgnnnnnnnnnnnnnnnnnnnnnntttgcgatgagacacnnnnnnnnnnnnnnnnnnnnagtccgggcaccccc

SEQ ID NO: 95 - DNA, 14 bp, synthetic construct
  misc_feature 1..14: note = First stem sequence within mir-144 hairpin
  source 1..14: mol_type = other DNA
tggggccctggctm

SEQ ID NO: 96 - DNA, 15 bp, synthetic construct
  misc_feature 1..15: note = Apical loop within mir-144 hairpin
  source 1..15: mol_type = other DNA
tttgcgatgagawmm

SEQ ID NO: 97 - DNA, 17 bp, synthetic construct
  misc_feature 1..17: note = Second stem sequence within mir-451 hairpin
  source 1..17: mol_type = other DNA
mwcttgctatacccaga

SEQ ID NO: 98 - DNA, 15 bp, synthetic construct
  misc_feature 1..15: note = Second stem sequence within mir-144 hairpin
  source 1..15: mol_type = other DNA
agtccgggcaccccc

SEQ ID NO: 99 - DNA, 72 bp, synthetic construct
  misc_feature 1..72: note = miRNA7 in miR-451
  source 1..72: mol_type = other DNA
cttgggaatggcaaggacagacttgcttaaaggaagtgtcctttaagcaagtctgctcttgctatacccaga

SEQ ID NO: 100 - DNA, 22 bp, synthetic construct
  source 1..22: mol_type = other DNA
tcaggtcttttcttgttcaccc

SEQ ID NO: 101 - DNA, 20 bp, synthetic construct
  source 1..20: mol_type = other DNA
acagacttgcttaaaggaag

SEQ ID NO: 102 - DNA, 23 bp, synthetic construct
  source 1..23: mol_type = other DNA
gaagtgaacaagaaagacctaac

SEQ ID NO: 103 - DNA, 18 bp, synthetic construct
  source 1..18: mol_type = other DNA
tcctttaagcaagtctgc

SEQ ID NO: 104 - DNA, 72 bp, synthetic construct; identical to SEQ ID NO: 99
  source 1..72: mol_type = other DNA

SEQ ID NO: 105 - DNA, 86 bp, synthetic construct
  source 1..86: mol_type = other DNA
tggggccctggctgtcaggtcttttcttgttcaccctttgcgatgagagaagtgaacaagaaagacctaacagtccgggcaccccc

SEQ ID NO: 106 - DNA, 483 bp, synthetic construct
  source 1..483: mol_type = other DNA
atcggcgctatgcttcctgtgcccccagtggggccctggctgtcaggtcttttcttgttcaccctttgcgatgagagaagtgaacaagaaagacctaacagtccgggcacccccagctctggagcctgacaaggaggacaggagagatgctgcaagcccaagaagctctctgctcagcctgtcacaacctactgactgccagggcacttgggaatggcaaggacagacttgcttaaaggaagtgtcctttaagcaagtctgctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 107 - DNA, 797 bp, synthetic construct
  source 1..797: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 108 - DNA, 133 bp, synthetic construct
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 109 - DNA, 101 bp, synthetic construct
  source 1..101: mol_type = other DNA
cccgtggcatccctgtgacccctccccagtgcctctcctggccctggaagttgccactccagtgcccaccagccttgtcctaataaaattaagttgcatca

SEQ ID NO: 110 - DNA, 1805 bp, synthetic construct
  source 1..1805: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 111 - DNA, 18 bp, synthetic construct
  source 1..18: mol_type = other DNA
gggaggtgtgggaggttt

SEQ ID NO: 112 - DNA, 20 bp, synthetic construct
  source 1..20: mol_type = other DNA
aatgattaacccgccatgct

SEQ ID NO: 113 - DNA, 25 bp, synthetic construct
  source 1..25: mol_type = other DNA
acttatctacagatctgcggccgct

SEQ ID NO: 114 - DNA, 22 bp, synthetic construct
  source 1..22: mol_type = other DNA
agtaacgccaatagggactttc

SEQ ID NO: 115 - DNA, 22 bp, synthetic construct
  source 1..22: mol_type = other DNA
ggcgtacttggcatatgataca

SEQ ID NO: 116 - DNA, 24 bp, synthetic construct
  source 1..24: mol_type = other DNA
ttacggtaaactgcccacttggca

SEQ ID NO: 117 - RNA, 23 nt, synthetic construct
  misc_feature 1..23: note = Reverse Transcription primer miRNA1
  source 1..23: mol_type = other RNA
tgctctttggtcttctcagccat

SEQ ID NO: 118 - RNA, 22 nt, synthetic construct
  source 1..22: mol_type = other RNA
ttgcccaactggtcctttttga

SEQ ID NO: 119 - DNA, 20 bp, synthetic construct
  source 1..20: mol_type = other DNA
aaatgttggaggagcagtgg

SEQ ID NO: 120 - DNA, 20 bp, synthetic construct
  source 1..20: mol_type = other DNA
tccagaattccttcctgtgg

SEQ ID NO: 121 - DNA, 21 bp, synthetic construct
  source 1..21: mol_type = other DNA
agcagggagcattgcagcagc

SEQ ID NO: 122 - DNA, 6575 bp, synthetic construct
  source 1..6575: mol_type = other DNA
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgctcgagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccgcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 123 - DNA, 6816 bp, synthetic construct
  source 1..6816: mol_type = other DNA
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgctcgaggctagccaagtgatgcccaagtcacaatagtcacttcctttaagcaagtctgtgtcatctcggagctgtgaagcaaccaggtcatgtcccacagaatggggagcacaccgacttgcattgctgccctcatatgcaagtcatcaccactctctagaagcttgggctgaaattgtgcaggccgccgccgccgccgccgccgggaagcccggggcccggatgcaggcaattccaccagtcgctagaggcgaaagcccgacacccagcttcggtcagagaaatgagagggaaagttgtttccctccttgttttcttctggttaatctttatcaggtcttttcttgttcaccctcagcgagtactgtgagagcaagtagtggggagagagggtgggaaaaacaaaaacacacacctcctaaacccacacctgctcttgctagaccccgcccccaaaagagaagcaaccgggcagcagggacggctgacacaccaagcgtcatcttttacgtgggcggaacttgtcgctgtttgacgcacctctctttcctagcgggacaccgtaggttacgtgcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 124 - DNA, 1837 bp, synthetic construct
  source 1..1837: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgtcaggtcttttcttgttcaccctttgcgatgagagaagtgaacaagaaagacctaacagtccgggcacccccagctctggagcctgacaaggaggacaggagagatgctgcaagcccaagaagctctctgctcagcctgtcacaacctactgactgccagggcacttgggaatggcaaggacagacttgcttaaaggaagtgtcctttaagcaagtctgctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcctgctttatttgtgaaatttgtgatgctattgctttatttgtaaccattataagctgcaataaacaagttaacaacaacaattgcattcattttatgtttcaggttcagggggaggtgtgggaggttttttaaagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 125 - DNA, 585 bp, synthetic construct
  source 1..585: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 126 - DNA, 86 bp, synthetic construct
  source 1..86: mol_type = other DNA
tggggccctggctgggatttcatcatatactgtaagtttgcgatgagacactacagtatagatgttgtactagtccgggcaccccc

SEQ ID NO: 127 - DNA, 86 bp, synthetic construct
  source 1..86: mol_type = other DNA
tggggccctggctgggatttcatcatatactgtaagtttgcgatgagacactacagtatagatcttgtactagtccgggcaccccc